(19) United States
(12) Patent Application Publication
(10) Pub. No.: US 2023/0024584 A1
(43) Pub. Date: Jan. 26, 2023

(54) ANTI-CANCER COMPOSITIONS AND METHODS

(71) Applicant: University of Kentucky Research Foundation, Lexington, KY (US)

(72) Inventors: Chengfeng Yang, Nicholasville, KY (US); Zhishan Wang, Nicholasville, KY (US); Yunfei Li, Lexington, KY (US)

(21) Appl. No.: 17/778,321

(22) PCT Filed: Nov. 20, 2020
(86) PCT No.: PCT/US20/61537

§ 371 Date: May 19, 2022

Related U.S. Application Data
(60) Provisional application No. 62/945,739, filed on Dec. 9, 2019.
(60) Provisional application No. 62/938,162, filed on Nov. 20, 2019.

(51) Int. Cl. A61K 47/69; A61K 31/4468; A61K 31/282; A61K 45/06; A61K 47/62; A61K 9/00

(52) U.S. Cl. CPC A61K 47/6929; A61K 31/4468; A61K 31/282; A61K 45/06; A61K 47/62; A61K 9/0053

(57) ABSTRACT

Treatments for cancer include miriplatin assembled into an ultra-small dot (uPtD) and/or lomitapide or a pharmaceutically-acceptable salt thereof. The uPtD and/or lomitapide can be encapsulated in a nanoparticle for administration to a subject. Some embodiments further include paclitaxel or a pharmaceutically-acceptable salt thereof.

### RELATED APPLICATIONS

This application claims priority from U.S. Provisional Application Ser. Nos. 62/938,162 and 62/945,739, filed Nov. 20, 2019 and Dec. 9, 2019, respectively, the entire disclosures of which are incorporated herein by this reference.

### TECHNICAL FIELD

The presently-disclosed subject matter generally relates to cancer treatment. In particular, certain embodiments of the presently-disclosed subject matter relate to inhibiting the growth of a cancer cell and to killing a cancer cell using platinum (II) and/or lomitapide, which can be encapsulated in a nanoparticle.

### INTRODUCTION

Triple-negative breast cancer (TNBC) usually shows more aggressive metastasis and worse prognosis than other breast cancer subtypes [1]. Unfortunately, no effective targeted therapies are currently available for treating TNBC, representing an unmet critical clinical need.

Cancer stem cells (CSCs) are considered as the seeds of cancer metastasis [2]. Eradication of cancer stem cells (CSCs) represents a novel therapeutic strategy against cancer metastasis. Circulating tumor cells (CTCs) possess many CSC-like properties [3], and it is known that advanced-stage breast cancer patients with high CTC counts after systemic anticancer therapy have poor clinical outcomes [4,5], suggesting that chemotherapy efficiency against CTCs may reflect the efficacy of systemic therapy [6]

Accordingly, there is a need in the art for additional compositions and methods for use in treating cancers, including cancers associated CTCs, as well as breast cancers, such as TNBC.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The use of platinum (II) genotoxic agents (Pt(II)) for breast cancer treatment has a long history dating back to early 1970's and they are widely used as the first-line therapy for TNBC [7]. However, less than 20% of TNBC patients could achieve a pathologic complete response after Pt(II) agents chemotherapy [8]. The effects of Pt(II) genotoxic drugs, including cisplatin and other Pt(II) analogues (e.g. carboplatin, oxaliplatin, NDDP, miriplatin), against CSCs are mainly dictated by the extent of DNA damages [9]. The two major downstream pathways of DNA damage response (DDR) are DNA damage checkpoints and apoptosis, both of which work in a coordinated way [10].

If DNA damage is repairable, checkpoints pathway will be partially activated, leading to cell-cycle arrest and DNA repair. Upon the completion of DNA repair, normal cell cycling resumes. This partially-activated DDR could potentiate the resistance of CSCs to genotoxic agents and the recovery potential of CSCs [11-14]. Thus, checkpoint activation is believed to coexist with suppression of apoptosis and the extent of DNA damage has an important impact on the fate of cancer cells including CSCs. The increase of DNA repair instead of apoptosis ultimately helps cancer cells to develop chemoresistance and metastasis.

In general, most of the commercial Pt(II)-based drugs trigger inadequate DNA damages, which may promote TNBC cells especially CSCs to become more tolerant to DNA damages. Such a tolerance may contribute to the development of TNBC metastasis, and resistance to chemo- and radio-therapy [11-14], for instance, the resistance seen in patients with breast cancer [15]. De novo drug discovery and development are particular challenging, and usually take 10-17 years from an idea to a marketed drug [16], but their success rates are lower than 10% [17]. This is especially true for the Pt(II) drug developments and only a handful of Pt(II) drugs are clinically available for now. One approach to increase success rate in new drug development is to exploit clinically-prescribed drugs with established safety profiles that may be effective for unanticipated indications.

Miriplatin is a lipophilic Pt(II) agent produced by Dainippon Sumitomo Pharma Co., Ltd. in Japan and was approved for hepatic artery administration after suspension in an oily lymphographic agent (Lipiodol Ultra-Fluide®) for local treatment of hepatocellular carcinoma in 2009. Miriplatin possesses very poor solubilities in organic solvents, which hinders miriplatin entrapment into nanoparticles (NPs) by conventional nanoprecipitation or thin-film hydration methods. To date, there have been no reports about the systemic administration of miriplatin.

Disclosed herein is a unique strategy for to synthesizing ultrasmall Pt(II) dots (uPtDs), having a diameter of approximately 1 nm. The transformation from free molecules to quantum dot-like structure facilitates nanoparticle (NP) encapsulation of miriplatin, via a nanoprecipitation method disclosed herein, and has the additional benefit of potentiating extra DNA-damaging capability of miriplatin, as also disclosed herein.

It was found that uPtDs NPs displayed a superior DNA damage capability via enhanced-interactions with DNA and a significantly stronger effect in reducing CSC-like property of TNBC cells, compared to conventional cisplatin and miriplatin. Mechanistically, the severe DNA damages induced by uPtDs NPs activated the CHK1/2-CDC25A-cyclin A/E pathway to induce cell cycle arrest. Moreover, uPtDs NPs can target the in vivo circulating tumor cells (CTCs) to suppress TNBC lung metastasis. Given the desired-safety profile of miriplatin, the uPtDs represent a promising therapeutic agent of the metal-based nanomedicines to reduce cancer metastasis.

The present invention is also based, in part, on the unexpected discovery that lomitapide has anti-cancer effects.

Lomitapide is a compound that was developed by Aegerion Pharmaceuticals (Cambridge, Mass.) and was approved by the U.S. Food and Drug Administration (FDA) for use as a lipid-lowering agent for treating familial hypercholesterolemia. Lomitapide achieves this utility by inhibiting microsomal triglyceride transfer protein (MTP), which is an enzyme needed for the synthesis of very low-density lipoprotein (VLDL) in the liver.

It was unexpectedly discovered that lomitapide displayed a strong cytotoxic effect on various types of triple negative breast cancer cells. It was also surprisingly discovered that lomitapide has a cytotoxic effect on other cancer cell lines, including multiple breast cancer cell lines. Furthermore, it was discovered that lomitapide treatment displayed a very potent inhibitory effect on cancer stem cell-like property in multiple cancer cell types

Accordingly, the presently-disclosed subject matter provides compositions and methods for use in treating cancers, including cancers associated with circulating tumor cells (CTCs) and breast cancers, such as triple negative breast cancer (TNBC).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. The successful conjugation of cRGD-SH with maleimide-PEG2000-DSPE characterized by 1H NMR spectra.

FIG. 2A-2C. The fabrication, characterization, and proposed mechanism of uPtDs NP. FIG. 2A: A schematic description of fabrication and characterization of uPtDs and uPtDs NP. The image of produced uPtDs (bright dots) was taken using a STEM under 200 kv. The uPtDs were encapsulated into NP to form uPtDs NPs. The images of uPtDs NPs were taken using a TEM under 80 kv. FIG. 2B: Representative HAADF-STEM images taken under 200 kv. Brighter nucleus was found at LM2 cells treated with 6.4 μM of uPtDs NPs for 3 h than these of cisplatin and miriplatin. The bright dots in STEM images represent Pt(II) dots. FIG. 2C: A schematic description of potential DNA interacting ways by free Pt(II) (left) and uPtDs (right).

FIG. 3. The representative image of miriplatin before (left) and after (right) being transformed into uPtDs.

FIG. 4A-4C. The size determination of uPtDs NPs and uPtDs by TEM and DLS methods. FIG. 4A: The size of uPtDs NPs shown in FIG. 2B determined by TEM is around 13 nm. FIG. 4B: The size of uPtDs NPs determined by DLS method is approximate 13 nm, which is consistent with the results from TEM analysis shown in (FIG. 4A). FIG. 4C: The size of uPtDs shown in FIG. 2B determined by TEM is around 1 nm.

FIG. 5. Subcellular location of uPtDs-vesicles in LM2 cells under HAADF-STEM. LM2 cells were treated by 6.4 μM of uPtDs NP and then chemically fixed, sliced and immobilized on the grid. UPtDs-loaded vesicles were found at the perinuclear area.

FIG. 6A-6B. Further Pt element validation for STEM and TEM images shown in FIG. 2A. FIG. 6A: The analysis of Energy Dispersive X-ray Spectroscopy (EDS) for STEM image in FIG. 2A confirms that the shining dots are Pt dots. FIG. 6B: The analysis of EDS for two TEM images in FIG. 2A confirms that the black dots are Pt dots.

FIG. 7. In vitro release profile of uPtDs NPs under pH5.5 and pH7.2. The results are expressed as mean±SD, n=3.

FIG. 8A-8C. The uPtDs NP treatment induces significantly stronger DNA damages and causes more cell cycle arrest in TNBC LM2 cells than free cisplatin or free miriplatin treatment. FIG. 8A: Representative pDNA images after agarose gel electrophoresis and EB staining. FIG. 8B: Quantitation and representative overlaid IF staining of DNA damage mark γ-H2A (red) and DNA DAPI staining (blue). The quantitated results are presented as percent of cells with γ-H2A positive staining per field of view (FOV) (mean±SD, n=27). * p<0.05, ordinary one-way ANOVA analysis. FIG. 8C: The representative histograms of flow cytometry analysis of cell cycle and the quantitated results. (mean±SD, n=3. * p<0.05, compared to negative control, cisplatin, miriplatin or NP blank treatment, two-way ANOVA analysis.).

FIG. 9A-9C. The uPtDs NP treatment exhibits a significantly stronger inhibitory effect on TNBC cell viability and growth than free cisplatin or free miriplatin treatment. FIG. 9A: MTT assay: MDA-MB-231, LM2 and SUM159 cells were treated with vehicle control, free cisplatin, free miriplatin, uPtDs NP, or same molar amount of blank nanoparticle (NP blank) for 72 h. At the end of treatment, cell viability was determined by MTT assay. The results are expressed as relative to vehicle control group (100%) and presented as mean±SD (n=7). * p<0.05, ordinary one-way ANOVA analysis. FIG. 9B: Clonogenic assay: One hundred of MDA-MB-231, LM2 or SUM159 cells were seeded into 6-cm dishes. After overnight culture, cells were treated with vehicle control, free cisplatin (6.4 μM), free miriplatin (6.4 μM), uPtDs NP (6.4 μM), or same molar amount of blank nanoparticle (NP blank) for 6 h and then cultured for 13 days. At the end of culture, cell clones were stained with crystal violet, photographed, and counted. The results are presented as mean±SD (n=3). * p<0.05, ordinary one-way ANOVA analysis. FIG. 9C: Soft agar colony formation assay: TNBC cells were treated with vehicle control, free cisplatin (6.4 μM), free miriplatin (6.4 μM), uPtDs NP (6.4 μM), or same molar amount of blank nanoparticle (NP blank) for 6 h and then collected for soft agar colony formation assay. The results are presented as mean±SD (n=3). * p<0.05, ordinary one-way ANOVA analysis.

FIG. 10A-10F. The DNA damage-CHK1/2-CDC25A-cyclin A/E pathway is greatly impacted by uPtDs NP treatment. FIG. 10A: Compared to non-dots-like Pt(II) genotoxic drugs, uPtDs NP causes more severe DNA damage revealed by Western blot analysis. FIG. 10B: The uPtDs NP treatment causes severe DNA damage in a dosage-dependent mode. FIG. 10C: Pharmacological inhibition of CHK1 (with 1, 5, 10 nM of CHIR124), ATM (with 5, 10, 20 μM of KU55933) or ATR (with 1, 5, 10 nM of AZD6378) partially reverses the effect of uPtDs NP treatment on the “CHK1/2-CDC25A-cyclin A/E” pathway in LM2 cells. FIG. 10D-10E: Inhibition of CHK1 reduces uPtDs NP treatment-caused DNA damage (FIG. 10D) and cell cycle arrest (FIG. 10E) in LM2 cells. Panel D shows the representative overlaid IF staining images of γ-H2A (red) and DNA DAPI (blue) in LM2 cells. * p<0.05, compared to negative control or uPtDs NP plus CHIR124 treatment group (mean±SD, n=3). (FIG. 10F) A schematic description of the model of the regulatory mechanism of DNA damage-CHK1/2-CDC25A-cyclin A/E pathway by uPtDs NP treatment.

FIG. 11. The uPtDs NP treatment exhibits a significantly stronger effect in inducing DNA damage and decreasing cell cycle-related protein expression in triple negative breast cancer cells than free cisplatin or miriplatin treatment. Representative Western blot images showing the expression levels of DNA damage- and cell cycle-related proteins. Triple negative breast cancer cells were treated with vehicle control, free cisplatin (6.4 μM), free miriplatin (6.4 μM), uPtDs NP (6.4 μM), or same molar amount of blank nanoparticle (NP blank) for 48 h and then collected for immunoblotting analysis.

FIG. 12. The numbers of γ-H2A staining positive cells per field of view counted under a fluorescent microscope in FIG. 10D (n=27). *: p<0.05, unpaired Mann-Whitney test.

FIG. 13A-13E. Systematic administration of uPtDs NP drastically diminishes TNBC lung metastasis. FIG. 13A: The brief experimental scheme. FIG. 13B: Images of mouse mammary xenograft tumors extracted from mice treated with cisplatin, uPtDs NP or NP blank, respectively. The endpoint was 7 weeks post-inoculation of LM2 cells. FIG. 13C: The tumor weights from each treatment group in panel (FIG. 13B) (mean±SD, n=6). ns, no significance was found among three groups, no pairing nonparametric one-way ANOVA analysis. FIG. 13D: The normalized ex vivo bioluminescence images of lungs harvested from the mice of each treatment group. FIG. 13E: The bioluminescence signal intensities from the ex vivo lung images in panel (d) (mean±SD, n=6). p values in (FIG. 13E) were calculated by one-way ANOVA analysis of no pairing nonparametric Kruskal-Wallis. * p<0.05.

FIG. 14. The representative images of immunohistochemistry staining against GFP expressed by LM2 cells. The samples are from the lungs of the mice treated by cisplatin, uPtDs NP and NP blank.

FIG. 15. The body weight monitoring of mice treated by cisplatin, uPtDs NP or NP blank respectively. No drastic difference was found among the three groups

FIG. 16A-16C. The uPtDs NP treatment exhibits a significantly stronger inhibitory effect on TNBC cell cancer stem cell-like property than free cisplatin or miriplatin treatment. FIG. 16A: Representative images of suspension spheres from each treatment group and the quantitated results of sphere formation assay. The results are presented as mean±SD (n=3). * p<0.05, ordinary one-way ANOVA analysis. Briefly, SUM159 cells were pretreated with 6.4 μM of cisplatin, miriplatin and uPtDs NP for 3 h. PBS was set as a negative control and NP blank was set as a control for uPtDs NP. Then cells were collected by trypsinization and resuspended in serum-free F-12 culture medium containing B-27 (1×), epidermal growth factor (20 ng/mL), basic fibroblast growth factor (20 ng/mL), heparin (5 μg/mL) and 1% of penicillin-streptomycin. The suspended cells were plated into the ultra-low adhesion 24-well cell culture plates (2500 cells per well) and cultured for 10 days for sphere formation. FIG. 16B: Western blot analysis of the expression levels of cancer stem cell markers. SUM159 cells were treated with vehicle control, free cisplatin (6.4 μM), free miriplatin (6.4 μM), uPtDs NP (6.4 μM), or same molar amount of blank nanoparticle (NP blank) for 48 h and collected for Western blot analysis.

FIG. 17. Capture and Cy5 identification of mimic CTCs in mouse peripheral bloodstream. RGD-LPH NPs utilized as uPtDs delivery platform could target more LM2 cells mimicking CTCs in peripheral bloodstream than free molecules. In flow cytometry examination, GFP expressing LM2 captured by RGD-LPH-Cy5 or free Cy5 will illustrate both GFP and Cy5 behavior while LM2 not being captured by Cy5 only illustrate GFP behavior. * p<0.05, two-tailed unpaired nonparametric Mann-Whitney t-test analysis.

FIGS. 18A-18D. The stronger in vivo inhibitory effect against CTC and lung metastasis by uPtDs NP treatment. FIG. 18A: The study scheme of examining highly-aggressive human LM2 cells on xenografted mouse. FIG. 18B: DNA electrophoresis results after PCR of total mouse blood DNA to analyze the human-specific 480 bp fragment Cr17_1aa/4bc of α-satellite DNA on chromosome 17 of LM2 cells. FIG. 18C: The relative Cr17_1aa/4bc expressions after being normalized by mouse fragment control Plak_WT. The band intensities from panel (FIG. 18B) were quantified by Image J. (n=4-5, * p<0.05, one-way ANOVA analysis of no pairing nonparametric Kruskal-Wallis test). FIG. 18D: QPCR analysis of human AP2XCZ6 in mouse blood using mouse actin as internal control. (n=5, * p<0.05, one-tailed unpaired Welch's t-test analysis).

FIG. 19. The uPtDs NP treatment shows limited efficacy against the progression of already-established lung metastatic foci. Briefly, 1 million TNBC LM2 cells were injected by tail vein on day 0. When lung metastatic foci were found on day 13 by IVIS imaging, uPtDs NP administration began at the dosage of 5 mg/kg, 5 mg/kg and 7.5 mg/kg per week and NP blank was set as the control. All mice were imaged and euthanized on day 49.

FIG. 20. A cartoon schematic description of the therapeutic index of uPtDs NP and stimuli-responsive NPs accumulating at tumor perivascular site having great associations with the release profiles.

FIGS. 21A-21F include data showing that lomitapide treatment significantly reduces breast cancer cell growth of various cell lines as determined by the MTT assay.

FIGS. 22A-22G include data reflecting that lomitapide treatment significantly reduces breast cancer cell growth as determined by the clonogenic assay. FIG. 22A includes representative images from each treatment groups, and FIGS. 22B-22G include the quantitation of clone numbers, compared to vehicle control treatment group.

FIGS. 23A-23D include data showing that lomitapide treatment significantly reduces breast cancer cell growth of various cell lines as determined by the soft agar colony formation assay. FIG. 23A includes representative soft agar images from each treatment groups. FIGS. 23B-23D include the quantitation of soft agar clone numbers, compared to vehicle control treatment group.

FIGS. 24A and 24B include data reflecting that lomitapide treatment significantly reduces cancer stem cell-like property of breast cancer cells. FIG. 24A includes representative sphere images from each treatment groups. FIG. 24B includes the quantitation of sphere numbers, compared to vehicle control treatment group.

FIGS. 25A-25D include data illustrating that lomitapide treatment causes significant G1 phase cell cycle arrest in breast cancer cells of various cell lines. Initially, 0.1˜0.3 million of breast cancer cells were seeded into 6 cm culture dish and cultured overnight; then, the culture media were switched into FBS free medium for serum-starvation for 24 h for cell synchronization. After synchronization, the culture media were changed into FBS-containing medium and vehicle control (DMSO) or lomitapide (final concentration: 5 μM) were added into corresponding dishes. After 24, 48 and 72 h culture, cells were harvested, fixed by ice-cold ethanol, stained by PI for flow cytometry analysis of cell cycle. The results are presented as mean±standard deviation (n=3). * p<0.05, compared to vehicle control (negative control) treatment group.

FIGS. 26A-26C include data showing that lomitapide treatment induces apoptosis of breast cancer cells.

FIGS. 27A-27E include data showing that lomitapide treatment significantly reduces lung cancer cell growth of various cell lines as determined by the MTT assay. Initially, 1000 of PC9, H1975, PC9GR4, H460, or A549 cells were seeded into 96-well plates. After overnight culture, vehicle (DMSO) or lomitapide (final concentration: 1.25, 2.5 and 5 μM) were added into the corresponding wells. After 24 h, 48 h and 72 h incubation, the culture media were aspirated off and 50 μL of 0.5 mg/mL of MTT containing medium was added into each well. After 3h incubation, 200 μL of DMSO were added into each well. The OD value of each well was read under the wavelength of 570 nm. The results are presented as mean±standard deviation (n=6) * p<0.05, compared to vehicle control (negative control) treatment group.

FIGS. 28A-28E include data reflecting that lomitapide treatment significantly reduces lung cancer cell growth as determined by the clonogenic assay. FIG. 28A includes representative images from each treatment groups. FIGS. 28B-28E include quantitation of clone numbers, compared to vehicle control treatment group.

FIGS. 29A and 29B include data showing that lomitapide treatment induces apoptosis of lung cancer cells.

FIGS. 30A and 30B include data establishing that lomitapide treatment significantly reduces cancer stem cell-like property of lung cancer cells.

FIGS. 31A-31F include data illustrating that lomitapide treatment significantly reduces colon cancer cell growth as determined by MTT assay.

FIGS. 32A-32D include data showing that lomitapide treatment significantly reduces colon cancer cell growth as determined by the clonogenic assay. FIG. 32A includes representative images from each treatment groups. FIGS. 32B-32D include quantitation of clone numbers, compared to vehicle control treatment group.

FIGS. 33A and 33B include data showing that systematic administration of nanoparticle-packaged lomitapide significantly reduces mouse orthotopic mammary tumor growth and spontaneous lung metastasis. FIG. 33A includes images of mouse mammary xenograft tumors and their weight extracted from mice treated with PBS, nanoparticle-packaged lomitapide or NP blank, respectively. FIG. 33 B includes normalized ex vivo bioluminescence images of lungs harvested from the mice of each group and the bioluminescence signal intensities from the ex vivo lung images.

FIGS. 34A-34C include data showing that systematic administration of nanoparticle-packaged lomitapide significantly reduces mammary tumor spontaneous lung metastasis in a syngeneic mouse model.

FIGS. 35A-35C include results showing the synergistic inhibitory effect of combined lomitapide (lomi) and paclitaxel (PTX) treatment on breast cancer cell growth at various doses.

FIG. 36 includes data showing the synergistic inhibitory effect of combined lomitapide (lomi) and paclitaxel (PTX) treatment on breast cancer cell growth in soft agar.

FIGS. 37A-37C include results showing the synergistic inhibitory effect of combined nanoparticle-packaged lomitapide (NP lomi) and paclitaxel (PTX) treatment on breast tumor growth and metastasis, including a brief treatment scheme on syngeneic 4T1 tumor model in FIG. 37A, ex vivo images of lungs with metastatic nodules in FIG. 37B, and weight comparison of the collected 4T1 primary tumors among groups in FIG. 37C.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compositions and methods for use in treating cancers. The presently-disclosed subject matter also includes methods for preparing compositions having anti-cancer utility.

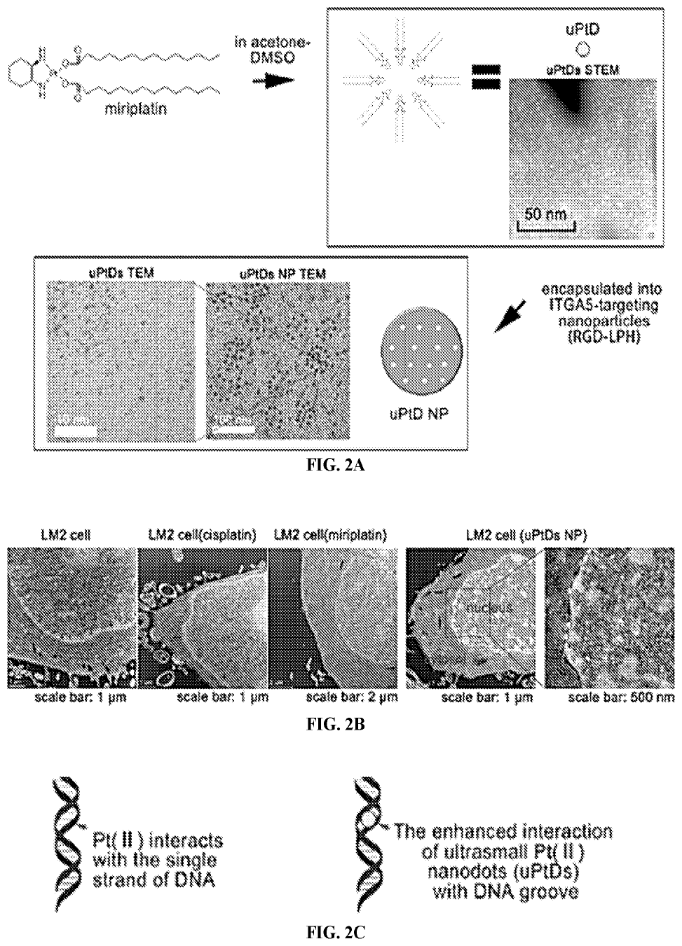

The presently-disclosed subject matter includes platinum (II) genotoxic agents (Pt(II)) assembled as ultrasmall dots. For example, in some embodiments, ultrasmall miriplatin dots (uPtDs) are provided. The dots are “ultra-small,” in that they have a diameter that beneficially allows them to fit into the DNA helix structure of cancer cells, to cause severe and/or lethal DNA damage to the cancer cells, such as double-stranded breaks (DSBs). See FIG. 2C. For example, in some embodiments, the diameter is less than about 2 nm. In some embodiments, the diameter is approximately 1.9, 1.8, 1.7, 1.6, 1.5, 1.4, 1.3, 1.2, 1.1, 1, 0.9, or 0.8 nm. In some embodiments, the diameter is approximately 1 nm. With reference to FIG. 2A, the assembly of the uPtD can be confirmed by visualizing the uPtD under STEM microscope (as a shining dot), while scattered miriplatin molecules cannot be so-visualized.

As disclosed herein, the uPtDs has anti-cancer utility. Accordingly, the presently-disclosed subject matter includes pharmaceutical compositions comprising the uPtDs, and methods for use thereof.

The presently-disclosed subject matter also relates to the surprising discovery that lomitapide has anti-cancer utility. It was further discovered that a combination of lomitapide and paclitaxel synergistically produce an anti-cancer effect. In this regard, the presently-disclosed subject matter includes pharmaceutical compositions comprising lomitapide or a pharmaceutically-acceptable salt thereof, or a combination of lomitapide and paclitaxel, or pharmaceutically-acceptable salts thereof, and methods for use thereof.

The compositions disclosed herein can further include, in some embodiments, an additional anti-cancer agent as is known in the art, or can be administered in a combination treatment with, or simultaneously with, an additional anti-cancer agent known in the art.

The presently-disclosed subject matter also includes compositions wherein the anti-cancer agent(s) is encapsulated in a nanoparticle. For example, in some embodiments, the uPtDs are encapsulated in a nanoparticle. For another example, in some embodiments, the lomitapide is encapsulated in a nanoparticle. For another example, in some embodiments, the lomitapide and paclitaxel are encapsulated in a nanoparticle.

As will be recognized by the skilled artisan upon studying this document, a variety of nanoparticles can be selected and used. Indeed, an nanoparticle with core-shell structure that is capable of loading the uPtD, lomitapide, and/or paclitaxel can be used. As will be appreciated, once the relevant anti-cancer agent(s) is encapsulated in the nanoparticle for delivery the composition would be stable, as evidenced by the exemplary nanoparticles shown in FIG. 2A.

In some embodiments, the nanoparticle can be a lipid-polymer hybrid (LPH) nanoparticle. As will be appreciated, in some embodiments, it can be useful to anchor the exterior layer of the nanoparticle with a polar species. As will also be appreciated, in some embodiments, it can be useful to decorate the exterior of the nanoparticle with a targeting agent. For example, in some embodiments, the exterior layer of the nanoparticle can be anchored with a polar species such as PEG₂₀₀₀. For another example, in some embodiments, the exterior layer of the nanoparticle can be decorated with a targeting agent to direct the nanoparticle to a target cell of interest. In this regard, for example, the targeting agent could be an RGD peptide for targeting triple-negative breast cancer (TNBC) cells.

The presently-disclosed subject matter further includes anti-cancer methods, such as methods of inhibiting the growth of and/or killing a cancer cell, which involve administering an effective amount of an agent or composition as disclosed herein, thereby inhibiting the growth of and/or killing the cancer cell.

The cancer cell can be any type of cancer cell. For example, in some embodiments, the cancer cell is a breast cancer cell, a lung cancer cell, or colon cancer cell. In some embodiments, the cancer cell is a triple-negative breast cancer cell.

In some embodiments of the method, the cancer cell is in a subject. In this regard, in some embodiments, the cancer cell is in a tumor. Accordingly, methods as disclosed herein include administering the composition to a subject, such as a human or other animal subject. In some embodiments, administering the composition reduces the tumor growth. In some embodiments, administering the composition reduces metastasis.

In some embodiments, the composition is administered to a subject in need of cancer treatment. In some embodiments, the subject has breast cancer, lung cancer, or colon cancer. In some embodiments, the subject has triple-negative breast cancer.

In some embodiments of the method, the composition can be administered by injection. In some embodiments, the composition comprises a uPtD, and is administered by injection. In some embodiments, the composition can be administered orally. In some embodiments, the composition comprises lomitapide, and is administered orally. In some embodiments, the lomitapide is administered at a dose of about 10 mg/kg body weight of the subject.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

### EXAMPLES

The presently-disclosed subject matter is further illustrated by the following specific but non-limiting examples. The following examples may include compilations of data that are representative of data gathered at various times during the course of development and experimentation related to the present invention.

### Example 1: Cell Lines, Chemicals and Animals

Human TNBC cell lines MDA-MB-231, LM2 and SUM159 cells were obtained and cultured as previously described[19]. The LM2 cell is a derivative of MDA-MB-231 cells, which was selected for its strong ability to metastasize to lung in vivo [20].

Miriplatin was purchased from MedChemExpress USA (Monmouth Junction, N.J.). Thia-zolyl Blue Tetrazolium Bromide (MTT), poly(lactic-co-glycolic acid) (PLGA, easter terminated, lactide:glycolide 75:25, M.W. 76,000-115,000) were purchased from Sigma-Aldrich (St Louis, Mo.).

PEG2000-DSPE was purchased from NOF corporation (Tokyo, Japan). RGD peptide with a terminal cysteine (c(RGDfC), M.W. 578.65, RGD-SH) was purchased from Peptides International, Inc. (Louisville, Ky.). Maleimide(mal)-PEG2000-DSPE was purchased from Jenkem Technology USA Inc. (Allen, Tex.).

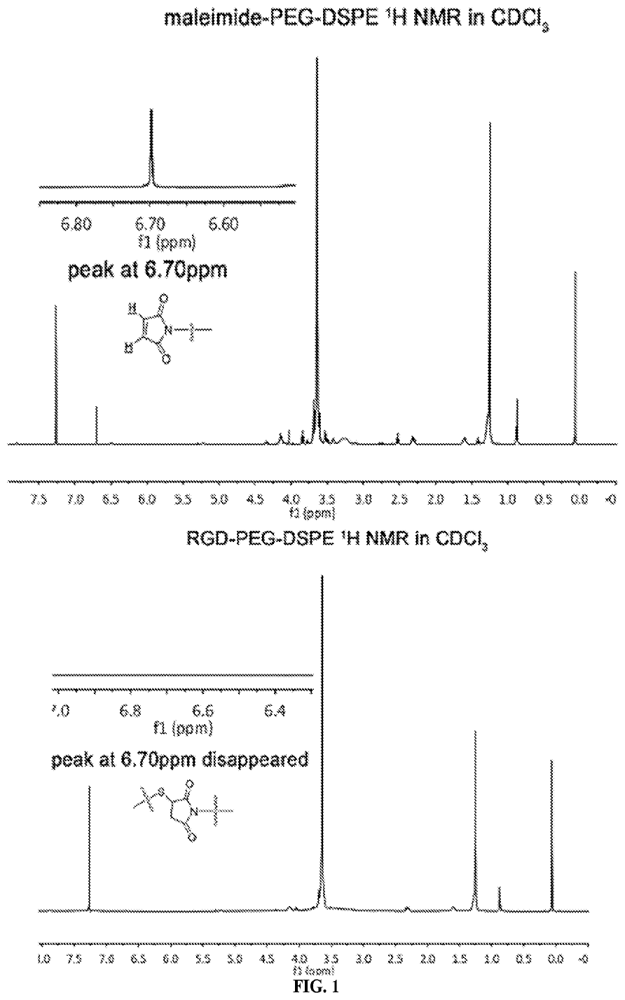

RGD peptide and mal-PEG2000-DSPE were used for the synthesis of RGD-PEG2000-DSPE by the published method [18] and the successful conjugation has been characterized by ¹H NMR (FIG. 1). Bioluminescence of lung metastases were quantitatively measured using IVIS Spectrum live animal imaging system (Perki-nElmer Co., Waltham, Mass.).

Nude mice (female, 6-8 weeks old at the beginning of each experiment) were purchased from the Charles River. All mice were housed in groups with unlimited access to food and water.

### Example 2: Preparation and Characterization of uPtDs NP

For the studies described herein, an ultrasmall Pt(II) dot (uPtD) was developed from miriplatin and encapsulated it into integrin α5 (ITGA5) active targeting nanoparticles (uPtDs NPs) and tested their therapeutic efficacy against TNBC metastasis.

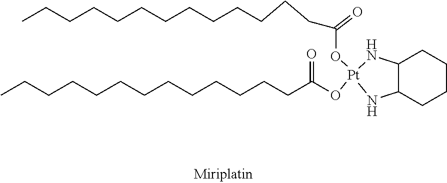

ITGA5 was previously found to be highly expressed in strongly migratory and invasive TNBC cells as well as their lung metastatic foci [18]. In this study, ITGA5 targeting lipid-polymer hybrid NPs [18] were used to produce uPtDs NPs, which renders lipophilic uPtDs water soluble and confers uPtDs active targeting capability.

Ultra-small miriplatin particles were prepared, having a diameter of approximate 1 nm, and are referred to as ultra-small miriplatin nanodots (uPtDs). These uPtDs can also be encapsulated in a nanoparticle. Aspects of encapsulation within a nanoparticle (NP) of an agent were previously described [18]. However, uPtD preparation, and subsequent encapsulation, has not been previously described.

For uPtDs preparation, first, 3 mg of miriplatin was suspended in 300 μL of DMSO and 700 μL of acetone, which was then heated in a water bath under 65° C. to a transparent solution.

Meanwhile, lecithin (2 mg), _{PEG2000-DSPE} (16 mg), and RGD-PEG2000-DSPE (2 mg) were dissolved in 30 mL of 4% ethanol aqueous solution and heated to 65° C. (Of course, other lipids could be selected of preparation of encapsulating nanoparticles, as desired, and as selected based on knowledge in the art about lipid nanoparticles).

Next, 1 mg of PLGA was mixed with miriplatin solutions followed by poured into the preheated lipid solutions, which then stood there still at room temperature to make sure the thorough self-assemble of uPtDs NP as well as the acetone evaporation.

Finally, the remaining free molecules (ethanol and acetone) were removed by an Amicon Ultra-4 centrifugal filter (Molecular cut-off 100,000, Millipore, Billerica, Mass.) and then resuspended in PBS to obtain a final desired concentration. The size (diameter, nm) of uPtDs NP was obtained using a Malvern Instruments Zetasizer ZS-90 instrument. The morphologies and sizes of uPtDs as well as uPtDs NP were observed utilizing transmission electron microscopy (TEM) under 80 kv and scanning transmission electron microscopy (STEM) under 200 kv (Talos, ThermoFisher Scientific, Hillsboro, Oreg.). NP blank was prepared by a similar method without the addition of miriplatin acetone-DMSO solution.

### Example 3: High-Angle Annular Dark-Field Scanning Transmission Election Microscopy (HAADF-STEM) Study of LM2 Cell-Epoxy Ultrathin Films

LM2 cells were seeded into 6 Well CELLSTAR® Cell Culture Multiwell Plates at a confluence of 50-70%. At the 2nd day, cisplatin, miriplatin and uPtDs NP (6.4 μM) were added into wells. After 6 h incubation, cells were fixed chemically by glutaraldehyde and osmium, dehydrated, embedded in epoxy resins, sliced and immobilized onto grids following common protocols but without the staining of salts of uranium and/or lead. HAADF-STEM under 200 kv (Talos, ThermoFisher Scientific, Hillsboro, Oreg.) was utilized to observe Pt element distributions inside the cells.

### Example 4: Determination of Miriplatin Contents and In Vitro Release Profile of uPtDs NP

A Varian Vista Pro CCD simultaneous ICP-OES was used to determine the concentration of Pt element in samples. Samples were digested with 0.5 mL aqua regia, then diluted with DI water to a total volume of 5 mL. This resulted in a 50× sample dilution prior to analysis. A 50 ppb analytical detection limit and a 2.5 ppm sample detection limit were established with platinum calibrations standards prepared in 5% HCl. Standard curve correlations maintained a corr. Coeff.>0.995. Sample measurements were read in triplicate. Quality control measures include a diluent blank, standard control, and yttrium internal standard measurements with each sample reading. Method conditions: Power (kW): 1.20; Plasma flow (L/min): 15.0; Auxiliary flow (L/min): 1.5; Nebulizer flow (L/min): 0.90; Replicate read time (s): 35.00; Instrument stabilization delay (s): 20. The miriplatin content was converted from Pt content by the following formula:

miriplatin content=(Pt content×Molecular weight of miriplatin)/(Molecular weight of Pt)

The weight ratio of miriplatin to NPs was defined as drug loadings (DL), while the weight ratio of entrapped miriplatin to added miriplatin was defined as entrapment efficiency (EE). Drug release over time was characterized by dialyzing samples against 1×PBS (pH 5.5 and 7.4) in 10 kDa MWCO Slide-A-Lyzer MINI dialysis units (Thermo Scientific) according to the reported methods [18]. The amount of drug retained in the NP samples was assessed at 0, 4, 12, 24, 48, 72 and 96 h and analyzed by method as described above.

### Example 5: DNA Electrophoresis to Study Interaction Between uPtDs NP and Plasmid DNA (pDNA)

Interactions of cisplatin, miriplatin, uPtDs NP and NP blank with pDNA (250 ng) were investigated by incubating 6 h and 12 h at 37° C. water bath. To make sure uPtDs inside the NPs fully exposed to the exterior environment, uPtDs NP were incubated overnight in PBS at 37° C. water bath while NP blank was used as a control. Samples were loaded into 2% agarose gel to determine the pDNAs shift. Naked pDNA and naked pDNA incubated at 37° C. water bath served as controls. The pDNA bands were visualized by EB staining and photographed using the Gel Doc EZ Imager (Bio-Rad).

### Example 6: MTT Assay, Clonogenic Assay, Soft Agar Colony Formation Assay and Suspension Serum-Free Culture Tumorigenic Sphere Formation Assay

The MTT assay, clonogenic assay, soft agar colony formation experiment and suspension serum-free culture tumorigenic sphere formation assay were carried out as described previously [18].

### Example 7: Western Blot Analysis, Immunofluorescence (IF) Staining and Immunohistochemistry (IHC) Staining

Cells were lysed using Tris-sodium dodecyl sulfate for Western blot analysis as previously described[18]. The following primary antibodies were used: cyclin A, cyclin E (Santa Cruz Biotechnology, Dallas, Tex.); p-CHK1, total CHK1, p-CHK2, total CHK2, CDC25A, c-myc, nanog, klf4 (Cell Signaling Technology, Beverly, Mass.); γ-H2A (Millipore Sigma, Burlington, Mass.); Cyclin D1 (BD Bioscience); and anti-β-actin (Sigma). The IF staining was performed following the previously described protocols [18]. The IHC staining against green fluorescent protein (GFP) in mouse lung tissues was performed following previously-described protocols [21].

### Example 8: Cell Cycle Analysis

LM2 cells were seeded into 6 cm dishes (density: 3×10⁵ cells per dish) and allowed to attach and grow overnight. After 24 h synchronization, cells were then exposed to 6.4 M of cisplatin, miriplatin, uPtDs NP or NP blank for 3 h respectively. After treatment withdrawal, LM2 cells were continued to culture 24 h. At end, cells were scrapped, washed, fixed and permeabilized with 70% ice-cold ethanol in 4° C. for 2 h. Cells were then incubated with freshly prepared propidium iodide (PI) staining buffer (0.1% Triton X-100, 200 μg/mL RNase A and 20 μg/mL PI in PBS) for 15 min at 37° C., followed by routine flow cytometry analysis of 20,000 cells in each group.

### Example 9: Evaluation on the Therapeutic Effect of Systemic Administration of uPtDs NP on TNBC Using a Nude Mouse Orthotopic Mammary Xenograft Tumor Model and an Experimental Lung Metastasis Model

Nude mouse orthotopic mammary xenograft tumors were established by directly injecting 1 million of LM2 cells into the 4th mammary gland fat pad as described previously [18]. The treatment started at day 14 post LM2 cell inoculation when mammary tumor volumes reached about 150-250 mm³ and terminated at day 49 post LM2 cell inoculation. Cisplatin, uPtDs NP and NP blank were administered thrice a week at the dosages of 2, 2, 3 mg cisplatin/kg body weight and 5, 5, 7.5 mg miriplatin/kg body weight [Pt(II) moles from cisplatin≈Pt(II) moles from miriplatin]; NP blank was set as a negative control. Tumor volumes were measured weekly. At day 49 post LM2 cell inoculation, all mice were sacrificed. The lungs were harvested and imaged by IVIS imaging individually using the same settings: exposure time 60s; F/Stop 1; Binning Small, Field of View D; all images captured were piled together and normalized by IVIS software. The mammary tumors were collected, weighted and imaged. For the experimental lung metastasis model experiment, one million of LM2 cells were injected via tail vein to each nude mouse to produce experimental lung metastasis at day 0. When lung metastatic foci were confirmed on day 13 by IVIS imaging, uPtDs NP administration began at the dosage of 5 mg/kg, 5 mg/kg and 7.5 mg/kg per week, and NP blank was set as the control. On day 49, all mice were imaged by IVIS machine to examine the bioluminescence intensity at thoracic area. After euthanasia, the lungs were collected and imaged ex vivo by IVIS machine as well.

### Example 10: Capture and Cyanine 5 (Cy5) Identification of Mimic CTCs in Mouse Peripheral Bloodstream

Nanovehicle-Cy5 were the same as RGD-LPH(Cy5), as previously described [18]. LM2 cells expressing GFP (0.5 million per mouse) were i.v. injected and 0.5 h later 300 μL of free Cy5 or nanovehicle-Cy5 solutions were i.v. injected into each mouse at the dosage of 0.6 mg Cy5/kg mouse body weight. Three hours later 0.5 mL of blood was drawn from abdominal artery by 25G syringe needle and spiked into 10 mL of RBC Lysis Buffer (BioLegend®) for 10 min incubation under room temperature. The pellets got after 10 min of centrifugation (1700 rpm) were resuspended by 0.5 mL of PBS with 2% FBS for flow cytometry examination. GFP expressing LM2 captured by nanovehicle-Cy5 or free Cy5 will present both GFP and Cy5 behavior while LM2 not being captured by Cy5 only illustrate GFP behavior.

### Example 11: PCR Method for the Detection of Human LM2 Cells in Xenotransplantation Mice

Nude mouse orthotopic mammary xenograft were established by the same above-mention method. At day 17 post-inoculation, the tumor sizes reached around 300 mm³ and treatment was scheduled trice per week at a dosage of 2 mg cisplatin/kg or 5 mg miriplatin/kg. At day 45 post-inoculation, around 0.5 mL mouse blood was collected from each mouse to MiniCollect® Tube (Greiner Bio-one GmbH, Austria) and then poured into 10 mL of 1×RBC lysis buffer (BioLegend®) and incubated for 10 min at room temperature for the removal of red blood cells. The lysis solutions were centrifuged under 1700 rpm for 10 min to collect the pellets, which will be further extracted DNA following the protocol from the manufacturer (DNeasy® Blood & Tissue Kit, QIAGEN GmbH, Germany). Then routine PCR reaction were performed and analyzed by DNA electrophoresis. The PCR reaction mixture (25 μL) contained 1 μL of dNTP (10 mM), 1 μL of each primer (10 μM), 1.5 μL of MgCl2 (50 mM), 2.5 μL of PCR Rxn buffer (10×, Invitrogen®), 1 μL of Taq DNA polymerase and 100 ng of genomic DNA template. Following an initial DNA denaturation and Taq activation at 94° C. for 10 min, 35 1-min cycles of denaturation at 94° C. and annealing/extension at 56° C. were performed followed by a final elongation step at 72° C. for 10 min. Oligonucleotides sequence (5′→3′) used for PCR:

forward primer(Cr17_1aa):
(SEQ ID NO: 1)
GGGTAATTTCAGCTGACTAAACAG;
reverse primer(Cr17_4bc):
(SEQ ID NO: 2)
AAACGTCCACTTGCAGATTCTAG;
forward primer(Plak_WT):
(SEQ ID NO: 3)
AACGATGAGGACCCGGTCTGAGAA;
reverse primer(Plak_WT):
(SEQ ID NO: 4)
TGGACAGCTGGTTCACGATCATAG.

### Example 12: The Human AP2XCZ6 qPCR Analysis

DNAs were extracted from the same mouse blood as indicated above. Human AP2XCZ6 primer was designed by Thermo Scientific based on 1-1000 of human alpha satellite from the centromeric region DNA with 16 monomer tandem repeats (Gene Bank Acc. No. 13882). AP2XCZ6 level analysis was carried out in ABI 7500 Fast Real Time PCR system and using mouse actin as the internal control for normalizing relative AP2XCZ6 expression level.

### Example 13: Statistical Analysis

Statistics were computed with GraphPad Prism 8 statistical software. The numerical data are expressed as mean±SD. A p value of <0.05 was considered statistically significant.

### Example 14: Results Associated with Physicochemical Characterization of ITGA5-Targeting NPs Loaded with uPtDs

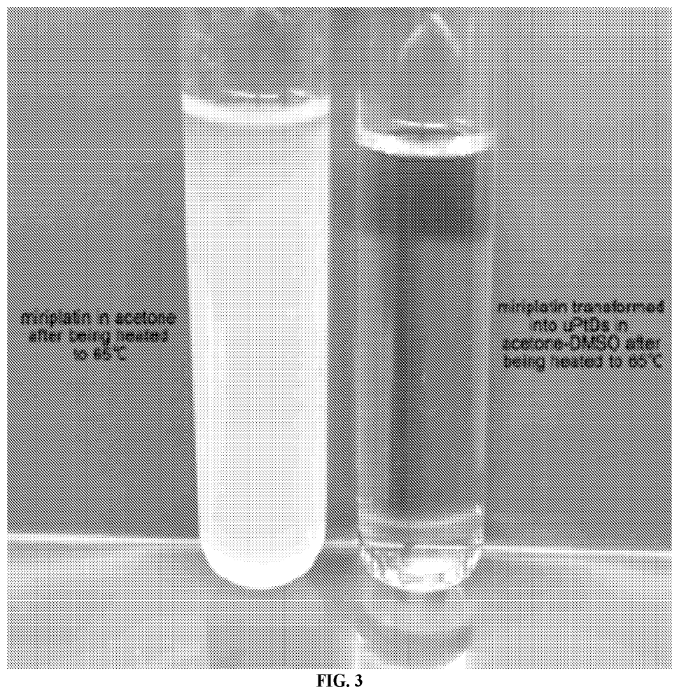

The fabricating approach of uPtDs NP was adapted from a previously-reported method [18], which is also briefly described in FIG. 2A. The solubility of miriplatin in acetone and other lipophilic organic solvents is unique. At room temperature or with heating, miriplatin was hardly soluble in acetone (FIG. 3) or other organic solvents. Interestingly, its suspension in acetone-DMSO (v/v, 7/3) turned into a transparent solution after being heated at 65° C. (FIG. 3) and formed uPtDs, which was confirmed by HAADF-STEM in FIG. 2A.

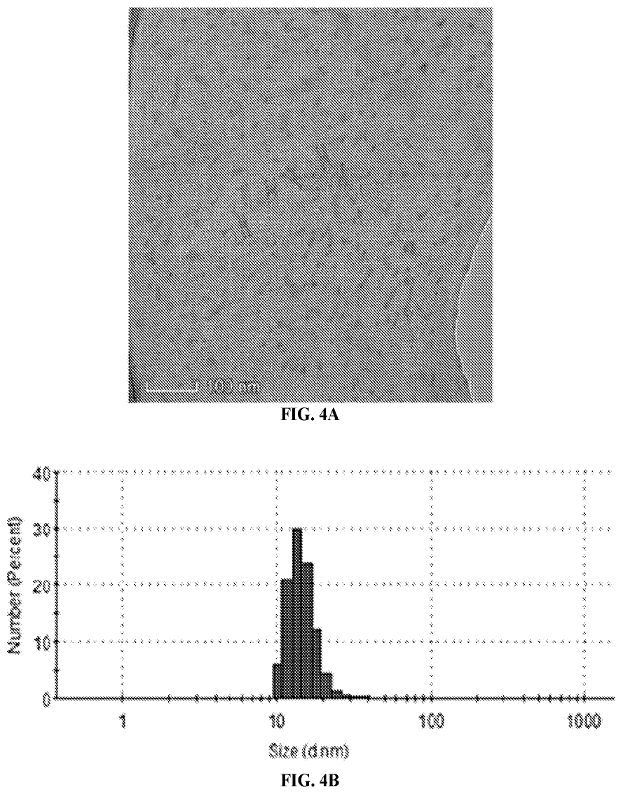

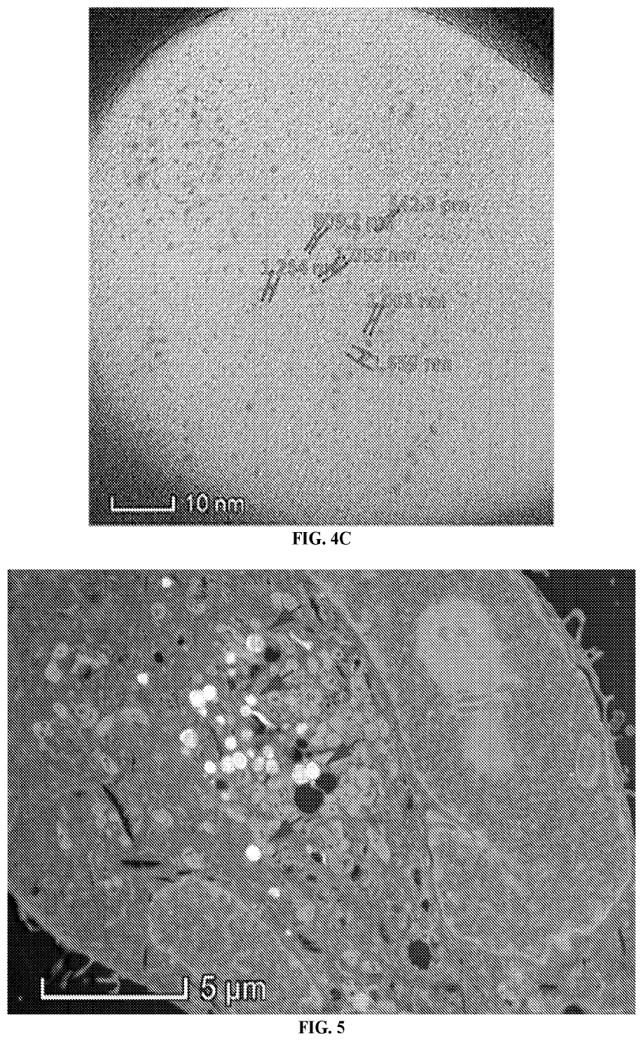

When pouring this uPtDs solution into an aqueous solution of lipids together with PLGA, uPtDs NPs self-assembled through nanoprecipitation. Examined by both dynamic light scattering (DLS) and TEM methods, uPtDs NPs were determined having diameters of 10-13 nm (FIGS. 4A and 4B). In TEM and STEM, only dots are visible whereas the individual Pt(II) ion is invisible (FIG. 2A). Specifically, in STEM of FIG. 2A, uPtDs present as a bright dot in a dark background.

FIGS. 2A and 4C show a piece of uPtDs, with a diameter of approximately 1 nm, which was smaller than the diameter of DNA major grooves (3.4 nm). Hence, uPtDs could readily fit into the grooves of DNA helices.

To explore whether uPtDs could enhance Pt(II) interactions with nucleus DNA in live cells, TNBC LM2 cell-epoxy sectioning was performed after exposure to 6.4 μM of uPtDs NP for 6 h, and then imaged as epoxy ultrathin sections under HAADF-STEM (FIG. 2B). The regions with brighter contrast in HAADF-STEM images indicate the presence of the heavier element Pt, while regions with lighter elements (e.g. P and O) produce a gray contrast.

As indicated by FIG. 2B, the brightness contrasts between cytosol and nucleus from LM2 cells treated by cisplatin or miriplatin share similar features as the control group without any treatments, whereas nucleus area of LM2 cell treated with uPtDs NP is drastically brighter than cytosol, indicating more extensive distribution of Pt in nucleus. Unfortunately, the direct evidence of uPtDs residing nucleus was not found by electron microscopy. According to FIG. 2B, uPtDs could have entered the nucleus but not be detected within the detection limit of HAADF-STEM. However, some vesicles filled with uPtDs resided in the perinuclear region (FIG. 5), which were consistent with previous studies involving subcellular location of nanovehicles [22, 23].

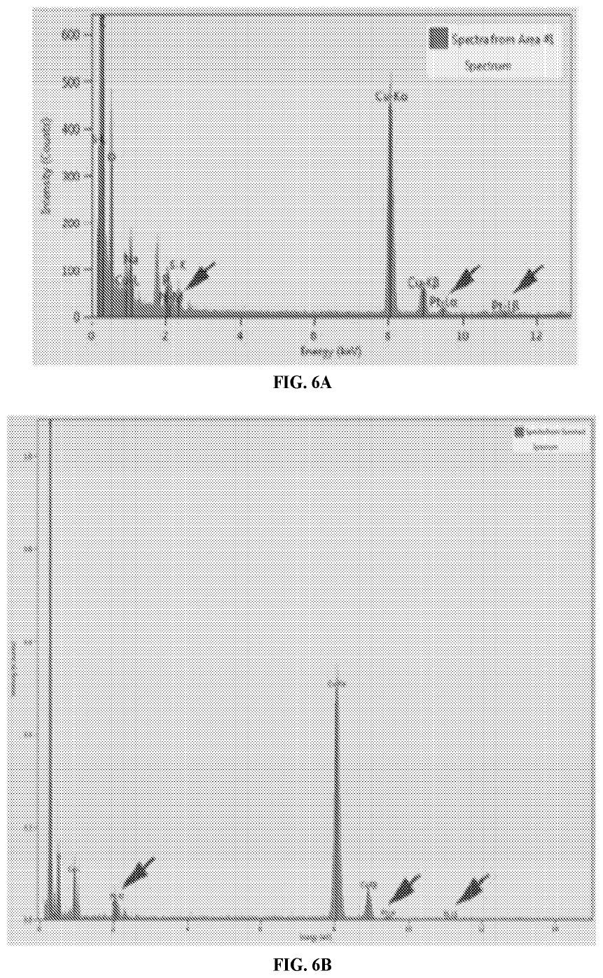

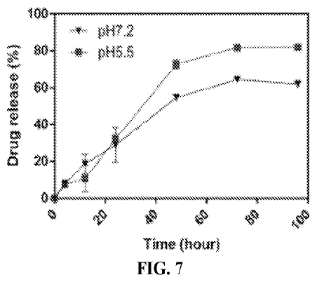

The bright distinctive spots of uPtDs NPs observed in STEM of FIG. 2A were evenly spread on copper grid surface, suggesting that the majority of uPtDs were well dispersed and retained their intact structures during the encapsulating process into NPs. As another modality of proof, the energy-dispersive X-ray spectroscopy (EDS) analysis under STEM mode (FIG. 6A-6B) further validated that uPtDs in STEM and TEM (FIGS. 2A, 4A and 4C) mainly consisted of Pt element. NPs (FIGS. 4A and 4B) are larger than the bare uPtDs in size as observed in TEM and STEM (FIGS. 2A and 4C) serving as another indication of successful NP encapsulation. The EE and DL capacity of uPtDs (calculated as miriplatin) into NPs were determined as 70.9±12.5% and 21.2±8.7%, respectively. Different release profiles between pH 5.5 and pH 7.2 were observed at 24-96 h, indicating that pH conditions could affect the release. However, the 48 h release profile under pH 7.2 was a sustained manner following zero order kinetics, which is indicative of robust NPs (FIG. 7).

Single or double-stranded DNA is known to interact with ultrasmall metal NPs such as gold, silver and Pt, to form nano-clusters [24,25]. Some hydrophobic substituents can help ultrasmall metal NPs fit into the non-polar DNA grooves leading to increased NP/DNA interactions [26], and Au NPs grafted with a hydrophobic monolayer have been applied in the gene transfection field [27]. Given the success of ultra-small metal NPs preferably interacting with DNA, spherical uPtDs with exterior hydrophobic myristates layer can potentially enhance DNA affinity (FIG. 2C) and lead to more DNA damages, serving as a potent genotoxic agent.

### Example 15: Results Associated with Increased DNA Interactions, Accrued DNA Damages and Cell Cycle Arrest by uPtDs NP Treatment

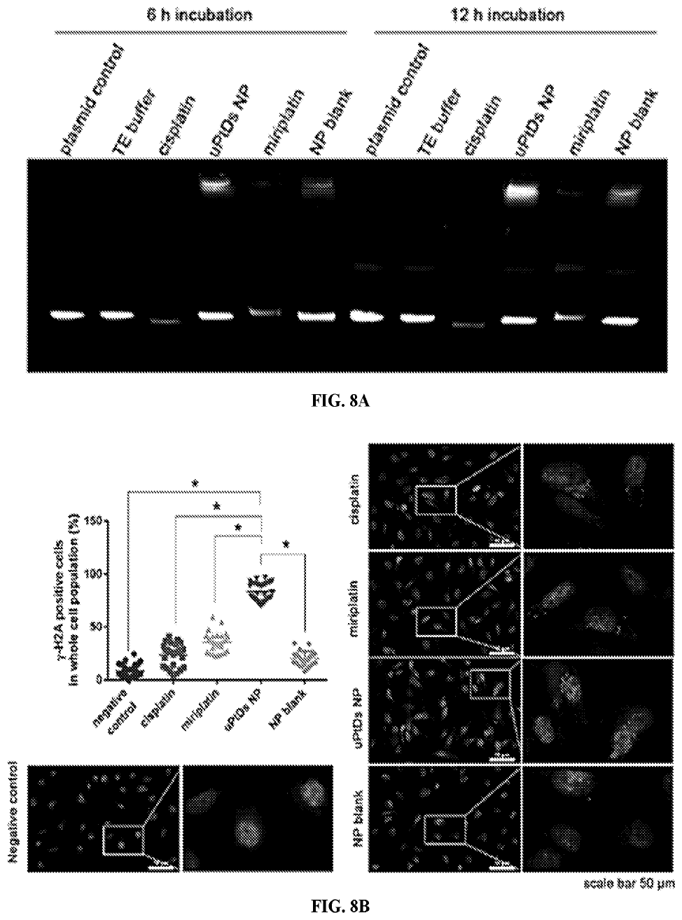

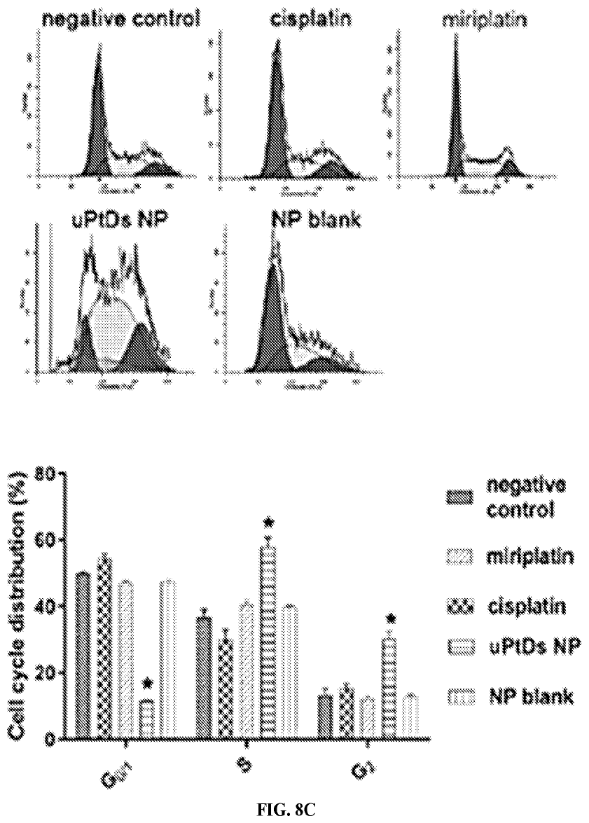

In vitro experiments were performed to explore uPtDs NP's abilities to interact with DNA (plasmid DNA used as the model DNA and hereinafter referred as pDNA) and damage DNA in TNBC LM2 cells. Gel electrophoresis in FIG. 8A shows the bands for cisplatin/pDNA, mir-iplatin/pDNA, uPtDs NP/pDNA (at the same Pt to DNA ratios) and NP blank/pDNA complexes. Of note, because of the sieving effect of the gel, the migrations of a large amount of uPtDs NP/pDNA complexes after 6 h and 12 h incubations were retarded as there were very bright bands found close to the loading wells. Moreover, the uPtDs NP/pDNA band after 12 h incubation was stronger than that of 6 h. No significant bands from the negative control, cisplatin and miriplatin lanes were found close to the loading wells. These results indicate that uPtDs had much stronger interactions with pDNA than cisplatin and miriplatin, which was probably due to binding of uPtDs to DNA grooves [24-26, 28, 29].

The intensities of migrating pDNA bands from cisplatin and miriplatin groups were relatively weaker than those of the other four lanes, which is because ethidium bromide (EB) bound to pDNA could be diminished by cisplatin during the incubation process [30]. Reportedly cisplatin could quench EB within 48 h [30]; and by the same way cisplatin and miriplatin quench EB bound to DNA in present study. Notably, when miriplatin was transformed into dots structure, Pt(II) resided in the dot core and were stable enough to avoid Pt(II) exposure during the incubation process. Due to this, EB quench phenomenon at uPtDs NP group was not observed. Interestingly, a much weaker band close to loading well was also found at NP blank group, suggesting blank NPs had some non-specific interactions with pDNA.

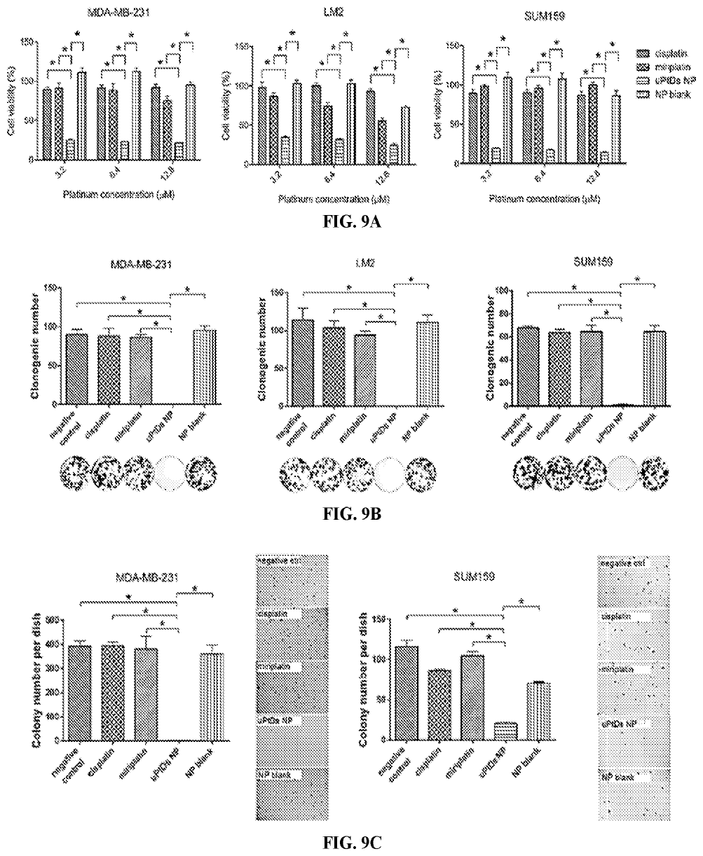

Next, IF staining of γ-H2A was performed to detect and compare the extent of DNA damage [31]. Due to their reported strong metastatic capability in vivo and inherent resistance to treatment [19,20], TNBC LM2 cells were chosen. In nearly 90% of cells treated with uPtDs NP, typical γ-H2A foci were observed 48 h post treatment (FIG. 8B). Conversely, significantly lower γ-H2A foci formations were observed in cells exposed to cisplatin, miriplatin or blank NPs (NP blank) (FIG. 8B). To repair the DNA damage, cell cycle checkpoints could be activated triggering cell cycle arrest. A significant portion (˜50%) of LM2 cells treated with cisplatin or miriplatin accumulated in _{G0/1} phase, which was similar to control cells (FIG. 8C). In sharp contrast, less than 15% of uPtDs NP-treated cells resided in the _{G0/1} phase, with a significant increase of cell population (˜85%) in S/G2 phase (FIG. 8C). The anti-proliferative study results (FIG. 9A-9C) are also in line with that from cell cycle analysis (FIG. 8C), both of which have a great association with the much stronger DNA damage effect of uPtDs.

### Example 16: Results Associated with DNA Damage Induced by uPtDs NP Activates the ATR/ATM-CHK1/2-CDC25A-Cyclin A/E Pathway

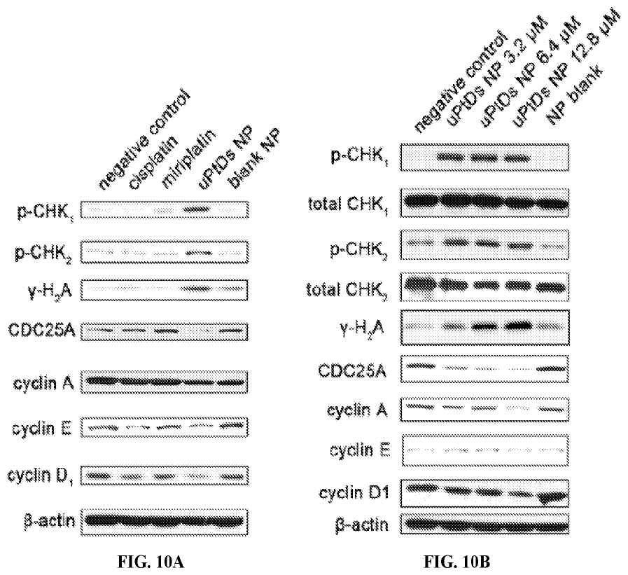

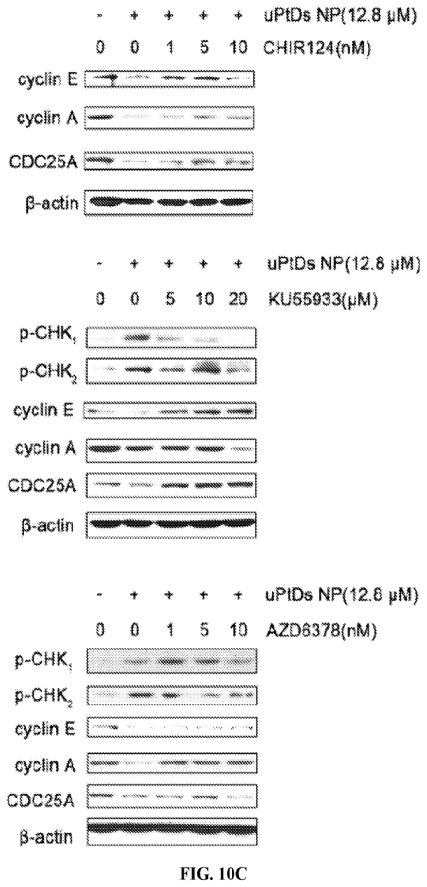

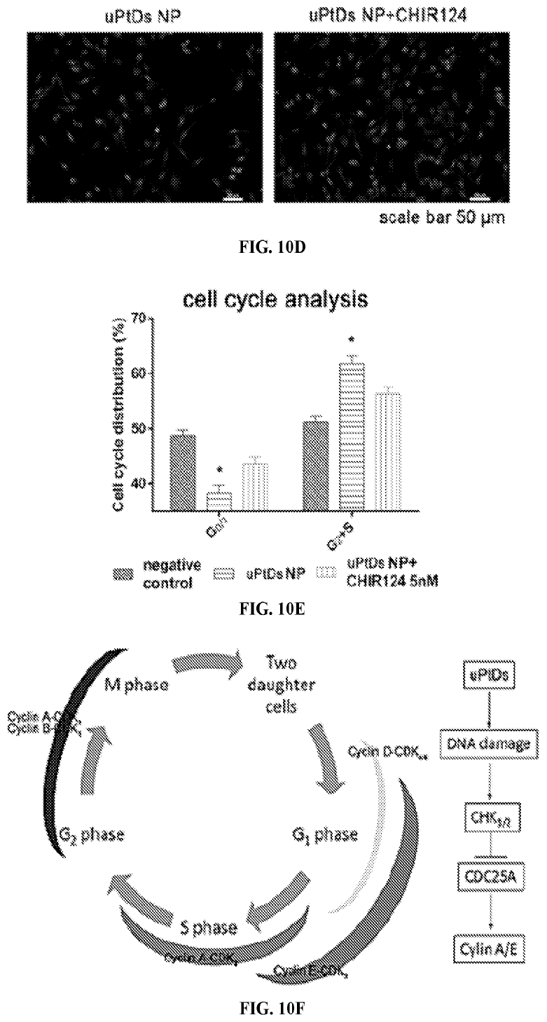

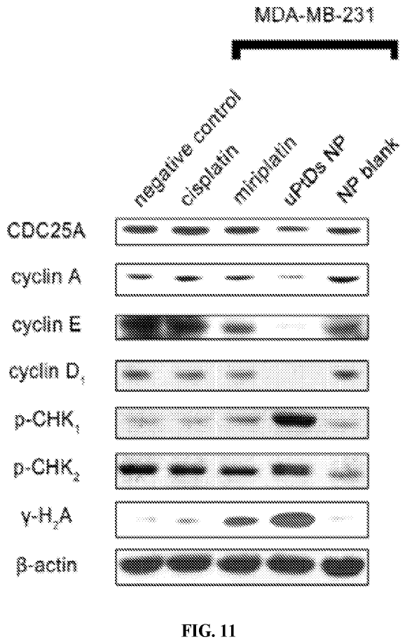

The underlying signal pathway was further elucidated. As shown in FIGS. 10A and 11, the expression of cyclin E and cyclin D1 decreased drastically after uPtDs NP treatment for 48h in LM2 and MDA-MB-231 cells. Cyclin A was slightly attenuated in LM2 and drastically attenuated in MDA-MB-231 cells. These results suggest that uPtDs NP causes cell-cycle arrest at S/G2 phases by attenuating cyclin D1, E and A expression levels. Next, as shown in FIGS. 10A and 11, drastically increased-levels of activated CHK1 and CHK2 (p-CHK1 and p-CHK2) were observed in LM2 and MDA-MB-231 cells treated with uPtDs NP. Consistent with this observation, the expression level of CDC25A was greatly decreased in uPtDs NP-treated LM2 and MDA-MB-231 cells (FIGS. 10A and 11). In line with the results in FIGS. 10A and 11, in a dose-dependent manner, a decrease in the levels of the CDC25A, cyclin A/D1/E proteins and an increase in the level of γ-H2A were detected by the treatment with uPtDs NP in the LM2 cells (FIG. 10B). An increase in the levels of the p-CHK1 and p-CHK2 proteins was also detected by the treatment with uPtDs NP, whereas total CHK1 and CHK2 levels were not altered (FIG. 10B).

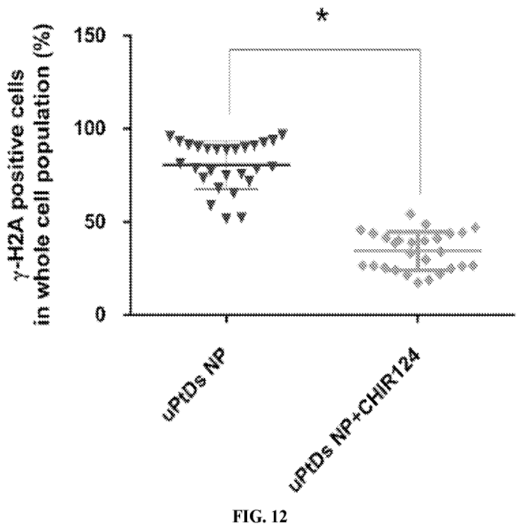

To further validate this DNA damage pathway, rescue experiments were performed utilizing CHK1(CHIR124), ATM (Ku55933) and ATR (AZD6378) inhibitors. By using pharmacological inhibition of CHK1, ATM or ATR activities, _{CHK1/2} were identified as the kinases that were activated by DNA damage and involved in uPtDs NP-induced down-regulation of CDC25A and cyclin A/E (FIGS. 10C-10F and 12).

### Example 17: Results Associated with uPtDs NP Impairing TNBC Cell CSC-Like Property and Targeting CTCs in Peripheral Bloodstream Reducing TNBC Tumor Lung Metastasis

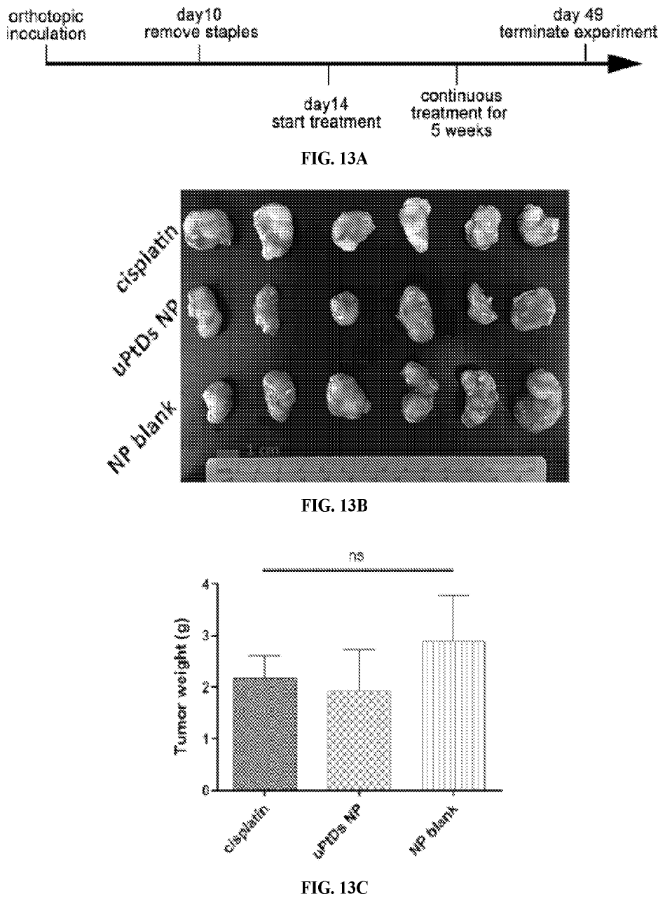

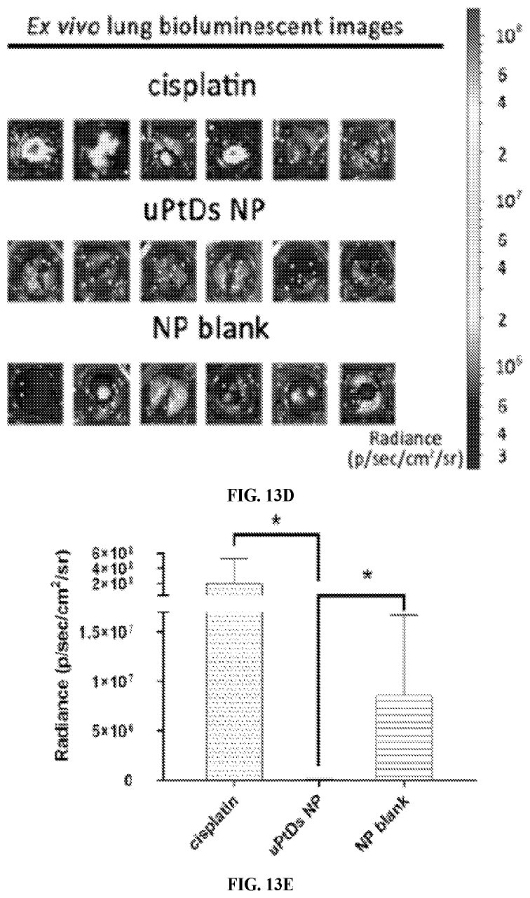

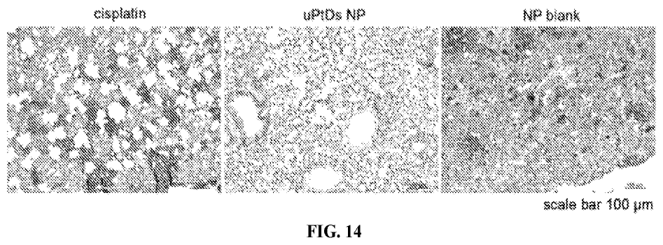

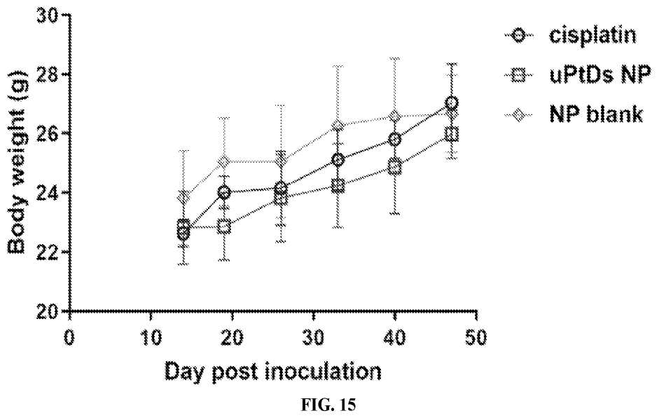

The in vivo efficacy of systematic administration of uPtDs NP on TNBC tumor growth and metastasis was then evaluated (FIG. 13A). It is striking that a 5-week uPtDs NP treatment via tail vein injection suppressed TNBC lung metastasis without impairing the primary tumor growth (FIG. 13B-13E). In contrast, mice treated with NP blank or cisplatin for 5 weeks still developed strong lung metastasis (FIGS. 13D-13E and 14). Moreover, the uPtDs NP treatment also had a good safety profile (FIG. 15), which was particularly interesting to us.

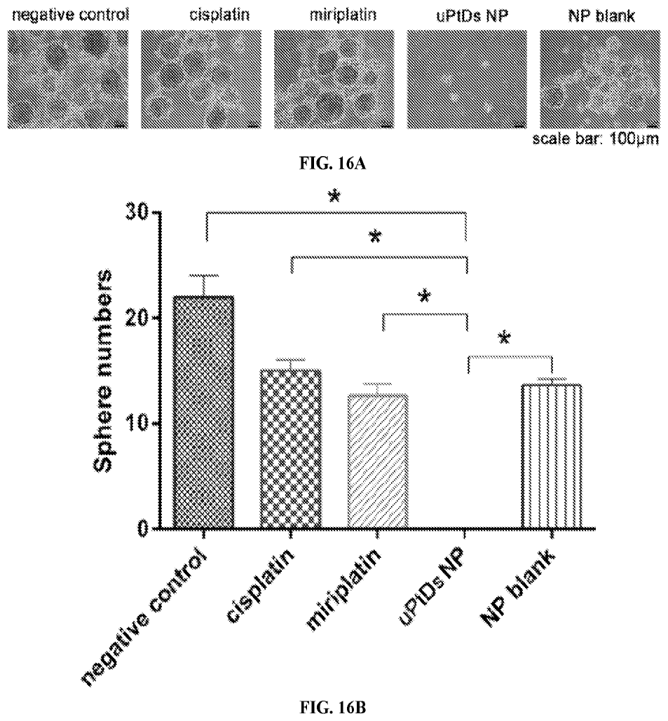

To investigate the mechanism of uPtDs NP treatment reducing TNBC metastasis, the effect of uPtDs NP treatment on TNBC cell CSC-like property was determined, as CSCs play crucial roles in metastasis. By using SUM159, another typical TNBC cells, a suspension and serum free culture tumorigenic sphere formation assay was performed. This is a well-established and widely-used assay for examining the presence of stem cell/CSC or CSC-like cells [32]. Because LM2 cells could not form spheroid structures under serum-free and suspension culture conditions, LM2 cells were not used for the sphere formation assay. As shown in FIG. 16A, uPtDs NP treatment drastically reduced the numbers of tu-morigenic suspension spheres formed by SUM159 cells. Additionally, the stemness-relating proteins in SUM159 cells after treatment were analyzed by immunoblotting (FIG. 16B). Despite that the c-myc levels from all five groups were consistent, the levels of nanog and klf4 greatly associated with CSCs [11,33] in uPtDs NP group were significantly attenuated compared to the other four groups. Both results in FIGS. 16A and 16B revealed that uPtDs NP could reduce significantly more TNBC CSC-like cells than free cisplatin or miriplatin treatment.

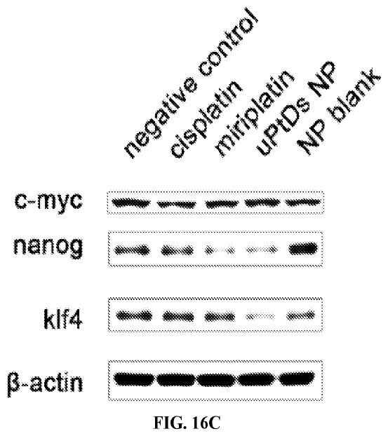

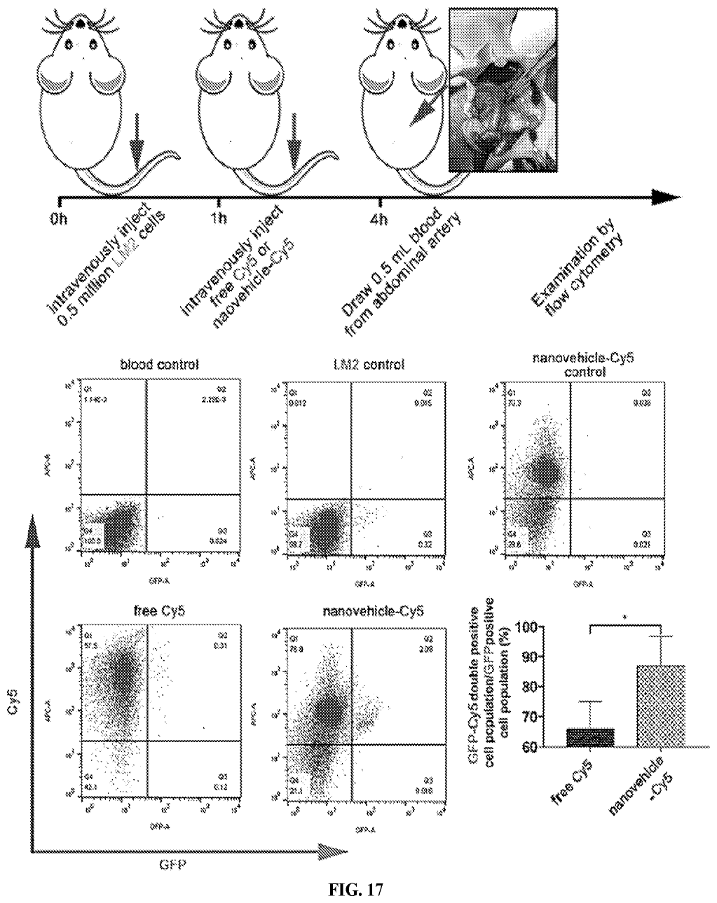

To further investigate the mechanism of uPtDs NP treatment reducing metastasis, the effect of uPtDs NP treatment on CTCs was determined, as CTCs are clinically considered as seeds of metastasis [4, 34-36]. However, the frequency of CTCs is extremely low in the complex peripheral bloodstream, and only up to hundreds of CTCs out of >10⁹ hematological cells were reportedly found in 1 mL blood [37]. Hereby, tail vein injection of 0.5 million LM2 cells expressing GFP to immunodeficient mouse was used to mimic CTCs. The studying scheme is briefly shown in FIG. 17. GFP-Cy5 double-positive cells are deemed as the CTCs targeted by free Cy5 or nanovehicle-Cy5 in bloodstream. As shown in FIG. 17, there are significantly more GFP-Cy5 double-positive cells in nanovehicle-Cy5 group (87.0±9.7%) than those in free Cy5 group (66.1±8.9%). This indicated that nanovehicles could deliver uPtDs to CTCs and reduce the number of CTCs more efficiently than free molecules such as cisplatin.

### Example 18: Results Associated with uPtDs NP Treatment Reducing the Presence of CTCs in Circulation of TNBC Tumor Bearing Mice

Furthermore, CTC levels were also determined in mice bearing LM2 xenograft mammary tumor with various treatment. Until now, there have been no in vivo studies using systemic administration of free miriplatin, since miriplatin is poorly soluble in most of the typical medical excipients such as Tween or Poloxamer. That is why the present study only utilized free cisplatin as the control. After orthotopic xenotransplantation of LM2 cells into mice, it is difficult to directly detect the CTCs that are present at a low level in peripheral bloodstream at treatment endpoint.

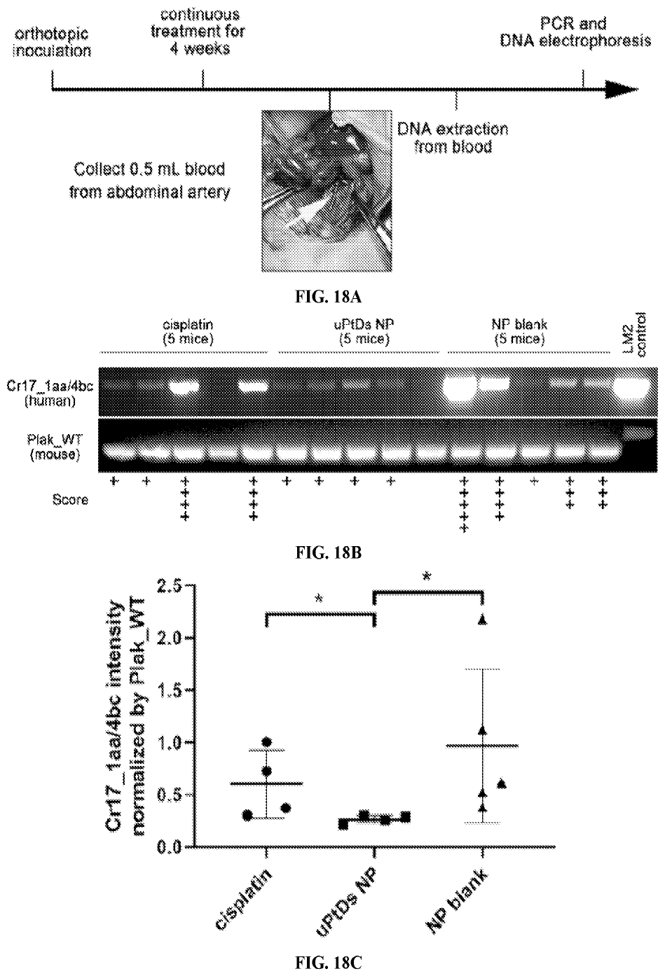

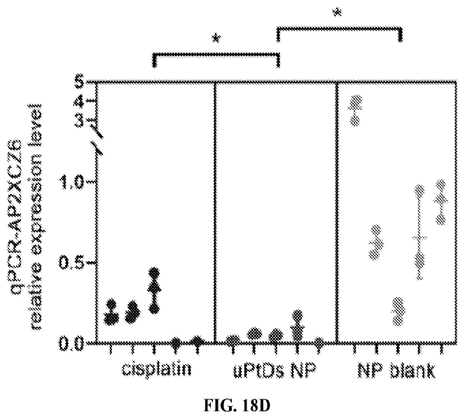

A reported sensitive PCR method was used to detect the presence of human cells (CTCs) in tumor bearing mouse blood circulation [38]. This method could detect a human specific 480 bp fragment of α-satellite DNA of the centromere region on human chromosome 17 (Cr17_1aa/4bc) in complex mouse tissue after xenotransplantation of malignant cells. The brief studying scheme is shown in FIG. 18A. At the end of the 4-week drug treatment (one week treatment less than the treatment in FIG. 13A-13E), 0.5 mL of abdominal artery blood was drawn for PCR analysis of Cr17_1aa/4bc, which was detected in 13 out of 15 mice in three experimental groups and the PCR band intensities was scored and expressed by one to five plus (FIG. 18B). One plus corresponded to very weak bands and five plus corresponded to very intense bands comparable to that of the positive control with 100% LM2 cells. Higher scores were recorded in cisplatin and NP blank-treated groups than these in uPtDs NP-treated group (FIG. 18B). Moreover, ImageJ software analysis of Cr17_1aa/4bc band intensities, which were normalized by mouse-specific DNA fragment control Plak_WT (FIG. 18C), and qPCR analysis (FIG. 18D) also showed that the blood from uPtDs NP treated mice had the significantly lowest LM2 level among the groups.

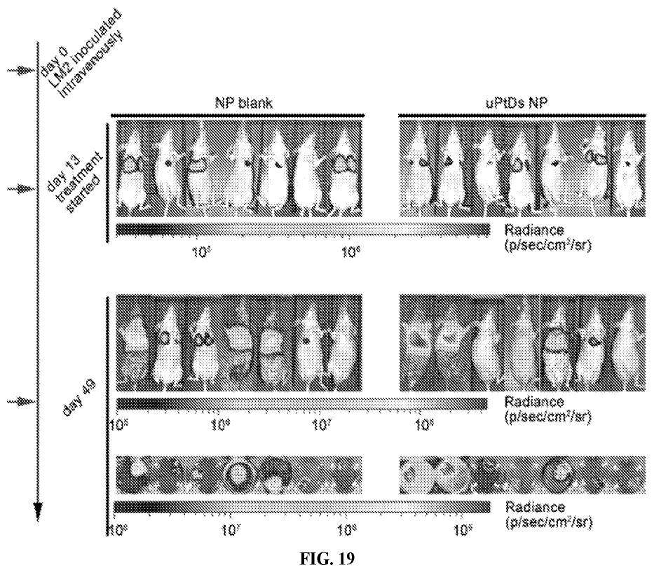

To further determine the mechanism of uPtDs NP treatment suppression of TNBC lung metastasis, the effect of uPtDs NP treatment on pre-existed (experimentally-induced) metastatic lung tumors was also investigated. As shown in FIG. 19, tail vein injection of LM2 cells was performed to produce lung metastatic tumor. Thirteen days after injection, lung metastatic tumors were formed in all mice as evidenced by the detection of chemiluminescence signals of all mouse lungs (FIG. 19). However, five weeks uPtDs NP treatment did not reduce or eliminate the chemiluminescence signals in mouse lungs. In fact, no significant difference of chemiluminescence signals in lungs of mice treated with uPtDs NP and blank NP was observed (FIG. 19). These results indicate that uPtDs NP treatment does not have a significant effect on the growth of already-existed metastatic lung tumors. Based on the results from FIGS. 13A-13E, 16A-16C, 17, 18A-18D, and 19, it is concluded that uPtDs NP could suppress TNBC lung metastasis mainly through decreasing CTCs, instead of reducing primary mammary tumor and metastatic lung tumor growth, which is distinct from most of current clinically-used therapeutic methods.

### Example 19: Discussion Related to Results of Examples 14-18

The fate of cancer cells following genotoxic insult is greatly impacted by the extent of DNA damage 39. Cisplatin is the most clinically successful DNA covalent binder. However, it usually leads to CSC-resistance due to its moderate DNA damage. Designing more Pt(II) complex molecules is the most popular strategy to enhance the efficacies of Pt(II)-based genotoxic agents but get limited success. There have been few studies on the therapeutic potentials of ultrasmall metal NPs (<5 nm), in one of which ultrasmall transition metal NP could trigger severe DNA damages as a radiosensitizer fitting into DNA helix grooves [40]. As shown in FIG. 2C, ultrasmall transition metal NPs have been reported to interact with DNA helix arising from groove fitting [24,25]. Of note, a study on Pt-metal-NPs emphasized the important role of Pt(II) ions blocking cell division by binding to DNA in DNA damage [41]. In another study, the limited effects of Pt-metal-NPs on DNA damage have been attributed to inadequate intracellular Pt(II) ions release [42]. Hence, both ultrasmall size and rapid generation/release of Pt(II) ions are essential for highly potent Pt-based NPs as genotoxic agents.

In the present study, uPtDs were prepared successfully starting from miriplatin. It is postulated that miriplatin could self-assemble into a sphere structure, the exterior of which were the myristates having good solubility in acetone and the core of which were the relatively lipo-phobic Pt(II) ions. However, the colloidal stability of uPtDs in aqueous environment is poor, due to their hydrophobic myristate exterior. To resolve this issue, additional coatings by NPs are needed. The NP encapsulation could help enhance the colloidal stability of uPtDs in aqueous environment. Because the threshold of renal clearance of NPs is ˜5.5 nm [43], NP encapsulation could help uPtDs (˜1 nm) avoid this problem. As expected, uPtDs were easily incorporated into ITGA5-tar-geting NPs through hydrophobic interactions. Under TEM and STEM, these Pt(II) ions were verified as ultrasmall Pt(II) dots with the diameter of around 1 nm (FIGS. 2A and 4A-4C). When uPtDs NP is endocytosed into the cell, the Pt(II) dots' structures are well preserved by the rigid shell composed of PLGA and lipids, which has been indicated by the vesicles containing uPtDs residing at the perinuclear area (FIG. 5). The exterior hydrophobic corona could facilitate the metal NPs fit into the non-polar DNA grooves leading to increased NP/DNA interactions [26, 27]. Similarly, the exterior hydrophobic myristates surrounding Pt(II) dots can potentiate uPtDs' affinity with DNA grooves (FIG. 2C) and lead to more DNA damages, serving as a potent genotoxic agent.

The present study found this ultrasmall dot prepared from mir-iplatin could cause severe DNA damages, possibly through the accumulation at nucleus (FIGS. 2b and 5) and the enhanced interactions with DNA grooves (FIGS. 2C and 8A). TNBC CSC fate is greatly impacted by the irreversible DNA damage by uPtDs (FIG. 16A-16C) disclosing their vast therapeutic potential. CTCs reportedly share stem-like mencaarkers and are regarded as tumor initiating cells or CSC-like cells [3,44], thus uPtDs NP is expected to impair CTCs as well. FIGS. 13A-13E, 16A-16C, 17, and 18A-18D revealed that NPs could target and reduce CTCs in vivo, which leveraged uPtDs' DNA-damaging advantages and thus suppressed TNBC metastasis in vivo. From a treatment landscape, eradicating the primary tumor and blocking the dissemination and targeting the metastatic foci are the three main ways to reduce tumor metastasis. The very limited efficacy of uPtDs NP against primary tumor (FIG. 13B) and pre-existed lung metastatic foci (FIG. 19) also indicated the great possibility of uPtDs NP reducing TNBC metastasis mainly via targeting CTCs.

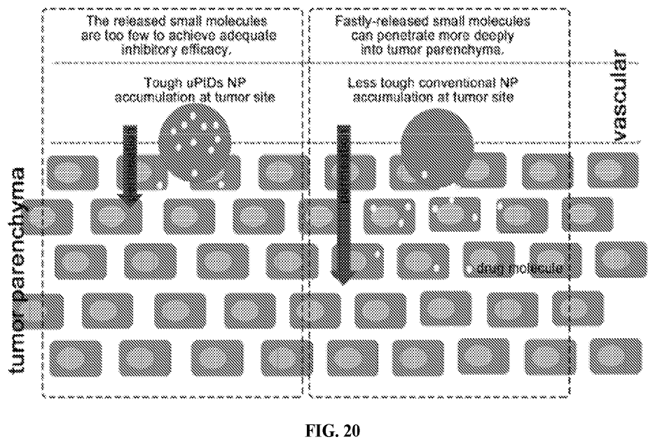

As for the reasons of uPtDs NP's inability to inhibit primary tumor growth, beside the intrinsic aggressive nature of LM2 cell-derived mammary tumor, the very extended release profile of the uPtDs NP (FIG. 7) may be another important reason. One study has disclosed that primary tumor cells endocytosed only 0.9% of the NPs that accumulate at the perivascular area of tumor [45], which suggest us that most of the NPs accumulating at tumor perivascular area may have to sufficiently release their payloads before exerting their therapeutic efficacies (FIG. 20). Generally, small molecules (<1 nm) have a better permeability than NPs (>10 nm), so the NPs with faster release profile outperform those with extended release profile after arriving at perivascular area of primary tumor as shown in FIG. 20. In a recent study, Yiguang Wang group designed a novel pH/cathepsin B double positive nanoparticle and successfully avoided the poor penetration problem, whereas the counterpart without stimuli-responsive release property only accumulated at tumor perivascular area [46]. In the present example, the uPtDs NP was not designed as a stimuli-responsive nanoparticle, and thus cannot intrinsically avoid the penetration problem by instantly-releasing the payload. Due to the slow release, not enough Pt(II) or dots are released to penetrate deeply into the tumor parenchyma to efficiently reduce primary tumor growth.

To gain further insights on the mechanism of superior therapeutic effects originating from enhanced DNA damages, the present study also examined the downstream signal pathway of the DNA damage elicited by this novel uPtDs and compared those by free cisplatin and mir-iplatin. Data in support of this conclusion were derived from several experimental approaches. First, as clearly indicated by γ-H2A IF staining shown in FIG. 8B, uPtDs caused more severe DNA damages whereas cisplatin and miriplatin only elicited moderate DNA damages. Second, CHK1/2 was activated by the severe DNA damage and involved in uPtDs-induced CDC25A and cyclin A/E downregulation (FIGS. 10A and 10B), leading to cell cycle arrest at S and G2 phase (FIG. 8C), which eventually impaired the proliferation and CSC-like properties of TNBC cells (FIGS. 9A-9C and 16A-16C). Third, validated CHK1 inhibitor (CHIR124) and ATM/ATR inhibitors (KU55933/AZD6378) prevented the attenuations of cyclin E, cyclin A and CDC25A by uPtDs NP treatment.

Collectively, the significant stronger inhibitory effect of uPtDs on tumor metastasis may be attributed to the following three factors. (1) The stability of uPtDs NP. The in vivo stability of nanomedicine is very important for the metastasis therapy, which has been comprehensively discussed by Omid C. Farokhzad et al. using Abraxane and Doxil as examples [47]. Both Doxil and Abraxane have very limited effect in improving patients' overall survival due to the rapid dissociation of payloads and excipients upon intravenous infusion [47]. Cheng Group reported anchoring hydrophobic polylactide (PLA) chains to paclitaxel molecule to avoid burst drug release profile and achieve higher drug loading of polymeric NPs [48]. Similarly, lipophilic corona of uPtDs formed by myristate chains in present study was well compatible with hydrophobic NPs core preventing the premature uPtDs clearance. The 48 h release profile of uPtDs NP was a sustained manner following zero order kinetics, which is indicative of robust NPs (FIG. 7).

(2) By virtue of their superior DNA damaging capabilities (FIGS. 8A-8C, 10A-10F, 9A-9C, and 11), uPtDs could overwhelm CTCs' DNA repairing abilities and reduce their amount (FIG. 16A-16C) and diminish CTCs more effectively than free cisplatin molecules.

(3) The β3 integrin family members (platelet _{αIIβ3}, tumor αvβ3) can also bind with RGD motifs, which have been anchored at uPtDs NP surface. Notably, the critical role of both _{αIIbβ3 and} tumor αᵥβ3 in CTC adhesion and invasion under blood flow conditions has been disclosed [49]; additionally, CTCs naturally prefer nanostructural surfaces [44] and NPs have large surface-to-volume ratio, both of which will enable efficient cellular binding by NPs.

The studies described in these Examples highlights the prominent DNA-damaging property of ultrasmall Pt(II) dots with the diameter of 1 nm in comparison to currently clinically-prescribed Pt(II) drugs. This strategy magnifies the DNA damage effects of Pt(II) atoms by leveraging the ultrasmall dot-like design architecture of miriplatin. In the meantime, all the excipients fabricating NP to encapsulate uPtDs are FDA-approved. Hence, uPtDs NP are contemplated to function as a safe and potent cancer chemo-therapeutic agent. While Pt(II) dot treatment and ITGA5-tar-geting NPs were used in the present study as proof-of-concept modalities, given the generalizability of ultrasmall dots design and their NP-encapsulating compatibility, it is envisioned that ultrasmall dot design strategy of Pt(II) atoms can be adapted toward a variety of other active metal complex against cancer metastasis and an alternative strategy for de novo development of metal-based drugs, in view of the information disclosed herein.

### Example 20: Lomitapide Treatment Reduces Growth of Breast Cancer Cells

A library of 1443 FDA-approved drugs was screened (Selleckchem, Cat. No. L1300). It was unexpectedly determined that lomitapide displayed a strong cytotoxic effect on three kinds of triple negative breast cancer cells. In particular, lomitapide treatment at a dose of 10 uM for 48 h reduced the viability of triple negative breast cancer in MDMA-MB-231-LM2, SUM-159 and MDA-MB-231 cells by 86.6%, 87.9%, and 87.4%, respectively.

It was surprisingly discovered that lomitapide has a cytotoxic effect on other cancer cell lines, including multiple breast cancer cell lines (MCF-7, MDA-MB-453, 4T1 and 4T1-luc), multiple lung cancer cell lines (A549, H460, PC9, PC9GR4, H1975), and multiple colon cancer cell lines (HCT 116, HCT29, DLD1, PT130 and SW480). By using MTT assay, clonogenic assay, soft agar colony formation assay and cell cycle analysis, lomitapide treatment at a dose of 2.5 or 5.0 μM for 72 h displayed strong inhibitory effects on cancer cell growth.

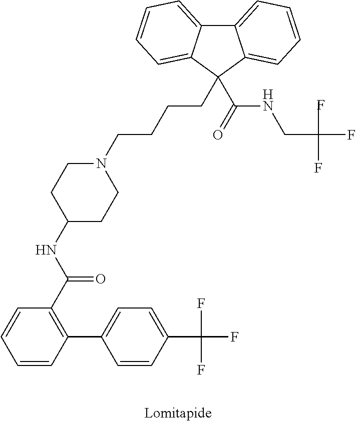

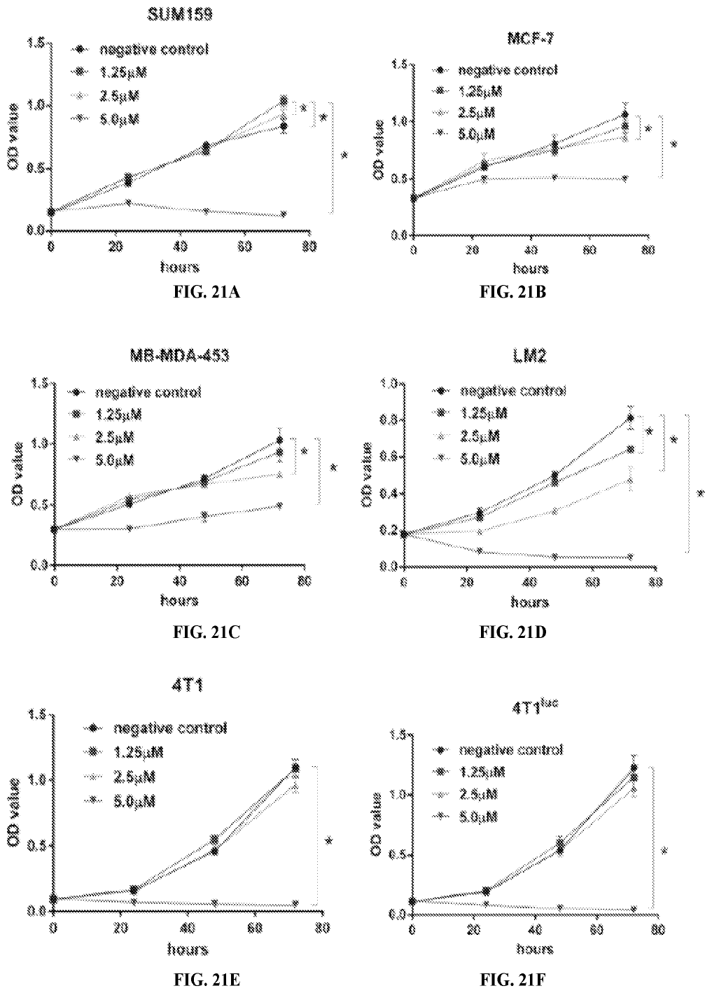

With reference to FIGS. 21A-21F, MTT assays were performed to determine the effect of lomitapide treatment on various breast cancer cell lines. Initially, 2500 SUM159 or LM2 cells, 5000 MCF-7 or MB-MDA-453 cells, 1000 4T1 or 4T1^{1uc} cells were seeded into 96-well plates. After overnight culture, vehicle (DMSO) or lomitapide (final concentration: 1.25, 2.5 and 5 μM) were added into the corresponding wells. After 24 h, 48 h, and 72 h incubation, the culture media were aspirated off and 50 μl of 0.5 mg/mL of MTT containing medium was added into each well. After 3 h incubation, 200 μl of DMSO were added into each well. The OD value of each well was read under the wavelength of 570 nm. The results are presented as mean±standard deviation (n=6) * p<0.05, compared to vehicle control (negative control) treatment group. These data show that lomitapide treatment significantly reduces breast cancer cell growth of various cell lines.

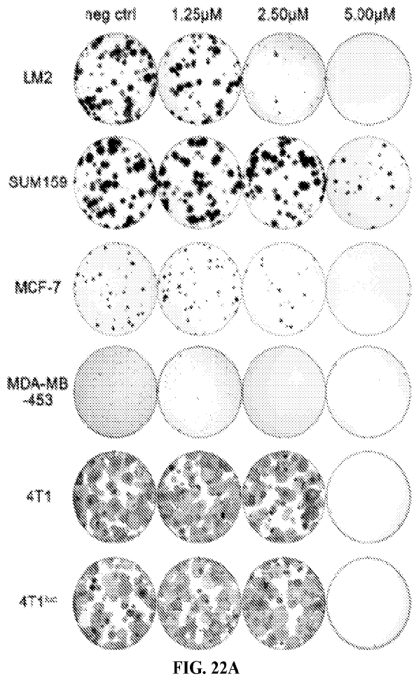

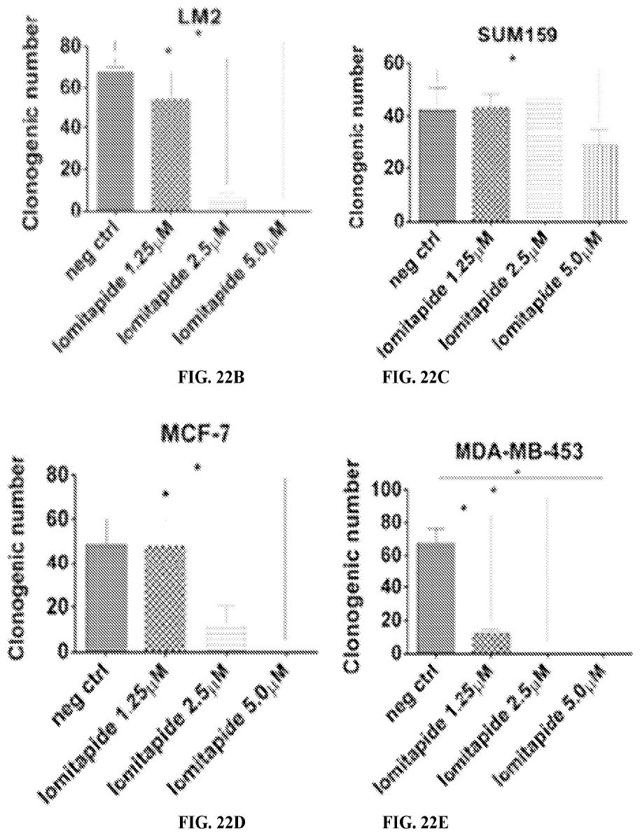

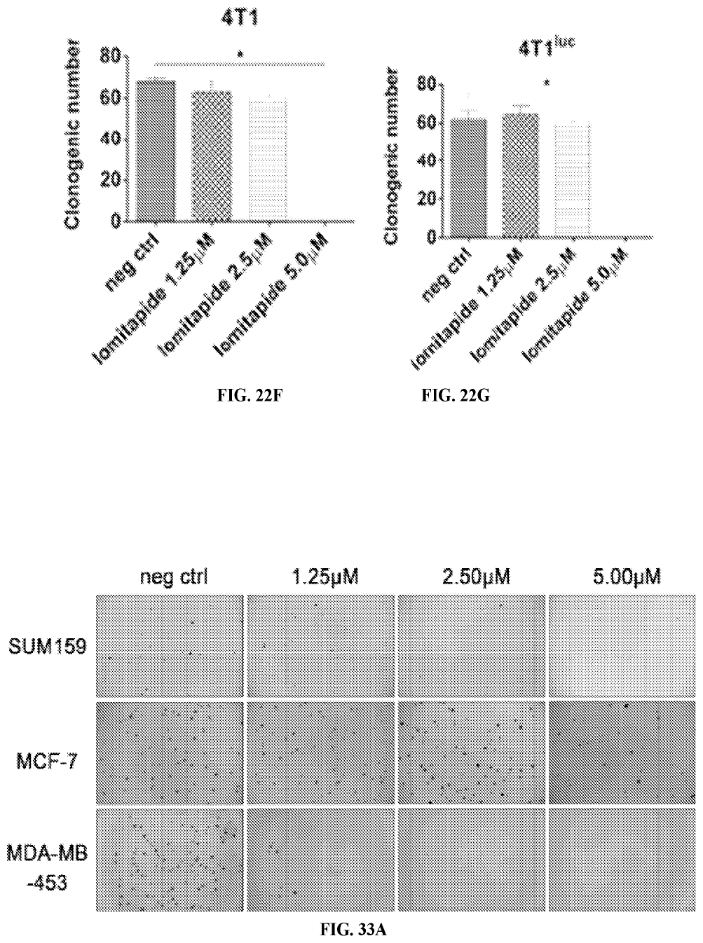

Clonogenic assays were also performed to determine the effect of lomitapide treatment on various breast cancer cell lines, with data reflected in FIGS. 22A-22G. Initially, 100 SUM159, LM2, MCF-7, 4T1 or 4T1^{luc} cells, or 1000 MDA-MB-453 cells were seeded into 6-cm cell culture dishes. After overnight culture, vehicle control (DMSO) or lomitapide (final concentration: 1.25, 2.5 or 5 μM) were added into the corresponding dishes. After 72 h culture, the DMSO- or lomitapide-containing media were removed and the fresh culture media were added in followed by additional 10 days culture. The culture media were replaced with fresh media every three days. At the end of cell culture, the final clones in dishes were fixed by 4% paraformaldehyde and stained with crystal violet. FIG. 22A includes representative images from each treatment groups, and FIGS. 22B-22G include the quantitation of clone numbers (mean standard deviation, n=3). * p<0.05, compared to vehicle control (negative control) treatment group. These data show that lomitapide treatment significantly reduces breast cancer cell growth of various cell lines.

With reference to FIGS. 23A-23D, soft agar colony formation assays were also performed. Initially, 1000 SUM159 or MCF-7 cells, 10000 MDA-MB-453 cells were seeded into the upper agar of each dish; meanwhile, vehicle control (DMSO) or lomitapide (final concentration: 1.25, 2.5 or 5 μM) were added into the upper agar of corresponding dishes. After 1 month continuous culture, the final colonies formed in the agar were fixed by methanol and stained with crystal violet. FIG. 23A includes representative soft agar images from each treatment groups. FIGS. 23B-23D include the quantitation of soft agar clone numbers (mean standard deviation, n=3). * p<0.05, compared to vehicle control (negative control) treatment group. These data show that lomitapide treatment significantly reduces breast cancer cell growth of various cell lines.

### Example 21: Lomitapide Treatment Reduces Cancer Stem Cell-Like Property of Breast Cancer Cells

Further studies were conducted using a suspension culture sphere formation assay, which is a well-established assay for analyzing cancer stem cell (CSC)-like property. It was discovered that lomitapide treatment displayed a very potent inhibitory effect on cancer stem cell-like property in multiple cancer cell types, including breast cancer and lung cancer cells.

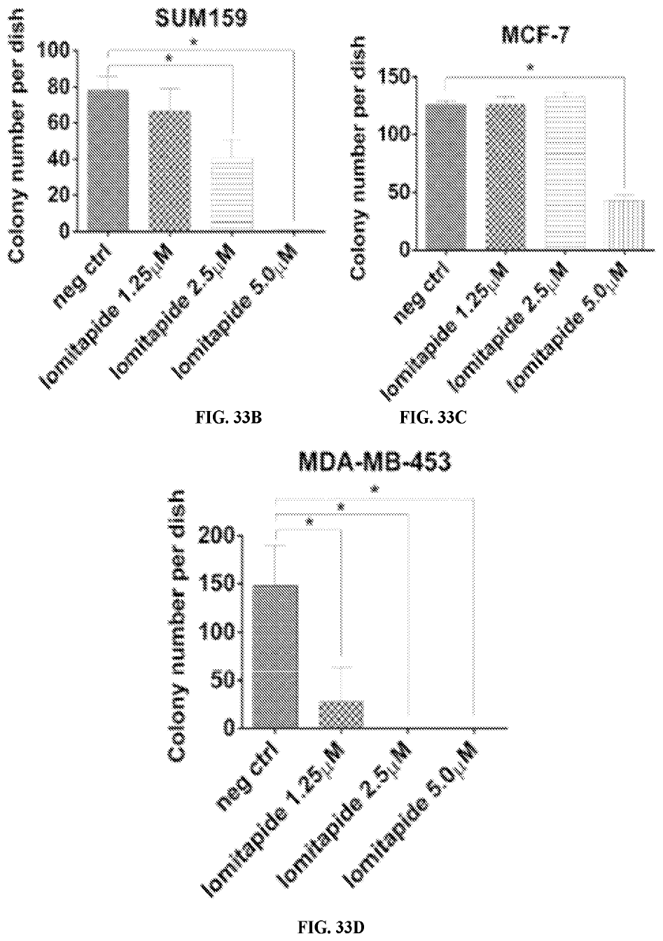

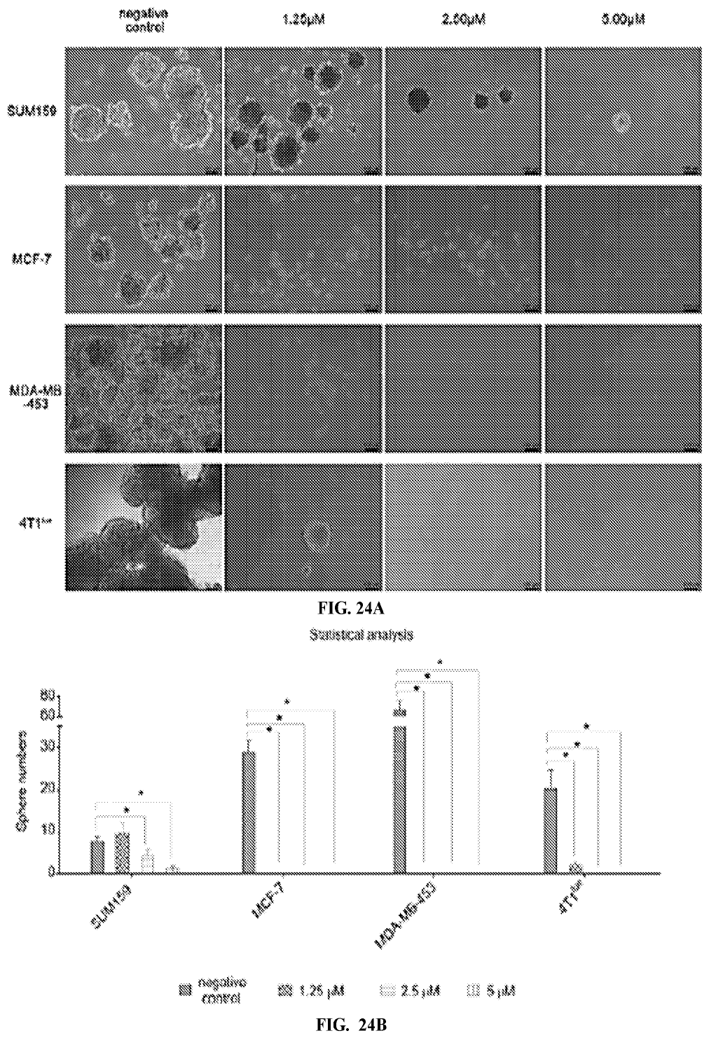

With reference to FIGS. 24A and 24B, the effect of lomitapide treatment on cancer stem cell-like property of breast cancer cells was further studied. Initially, 2500 breast cancer SUM19, MCF-7, MDA-MB-453 or 4T1^{luc} cells were seeded into ultra-adherance 24-well plates; meanwhile, vehicle control (DMSO) or lomitapide (final concentration: 1.25, 2.5 or 5 μM) were added into corresponding wells. After 10-days continuous culture, the final spheres formed were imaged and counted under an inverted phase-contrast microscope. FIG. 24A includes representative sphere images from each treatment groups. FIG. 24B includes the quantitation of sphere numbers (mean±standard deviation, n=3). * p<0.05, compared to vehicle control (negative control) treatment group. These data reflect that lomitapide treatment significantly reduces cancer stem cell-like property of breast cancer cells

### Example 22: Lomitapide Treatment Causes G1 Phase Cell Cycle Arrest in Breast Cancer Cells

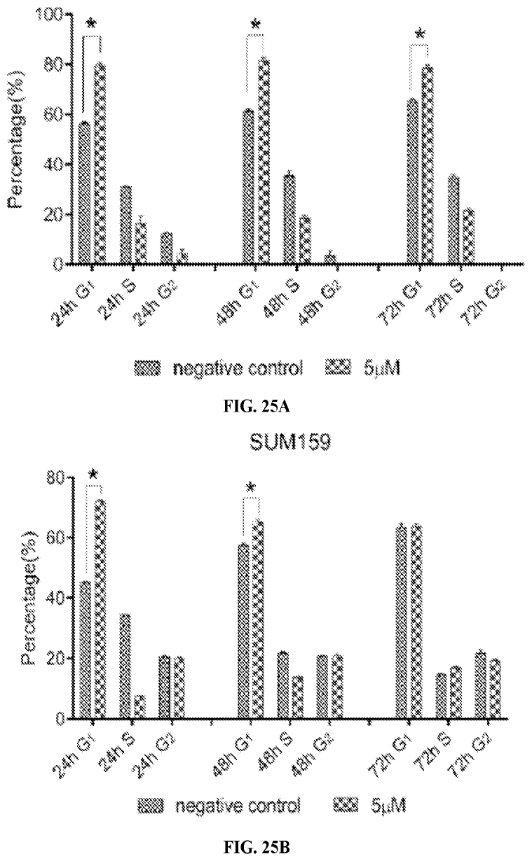

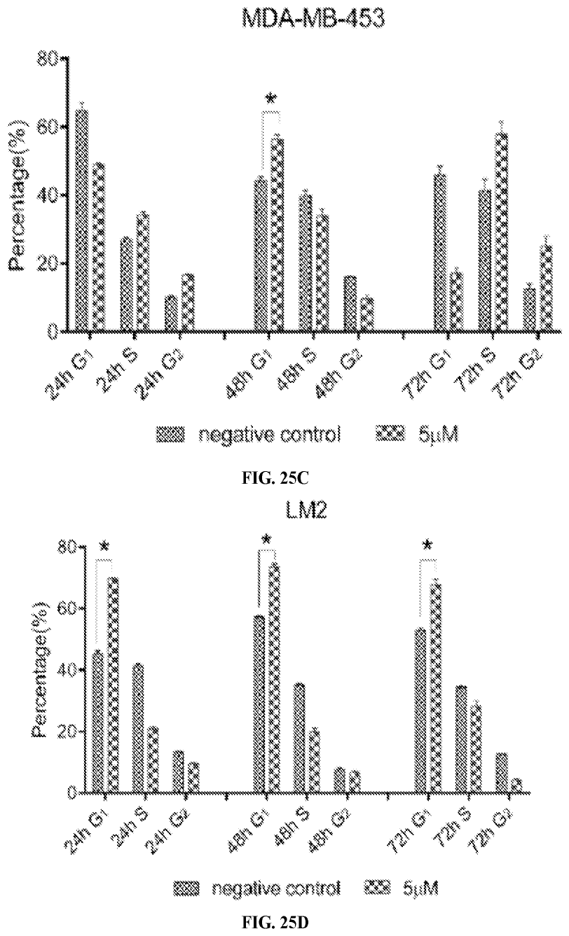

With reference to FIGS. 25A-25D, the effect of lomitapide treatment on significant G1 phase cell cycle arrest in breast cancer cells of various cell lines was also studied. Initially, 0.1˜0.3 million of breast cancer cells were seeded into 6 cm culture dish and cultured overnight; then, the culture media were switched into FBS free medium for serum-starvation for 24 h for cell synchronization. After synchronization, the culture media were changed into FBS-containing medium and vehicle control (DMSO) or lomitapide (final concentration: 5 μM) were added into corresponding dishes. After 24, 48 and 72 h culture, cells were harvested, fixed by ice-cold ethanol, stained by PI for flow cytometry analysis of cell cycle. The results are presented as mean±standard deviation (n=3). * p<0.05, compared to vehicle control (negative control) treatment group. These data illustrate that lomitapide treatment causes significant G1 phase cell cycle arrest in breast cancer cells of various cell lines.

### Example 23: Lomitapide Treatment Induces Apoptosis in Breast Cancer Cells

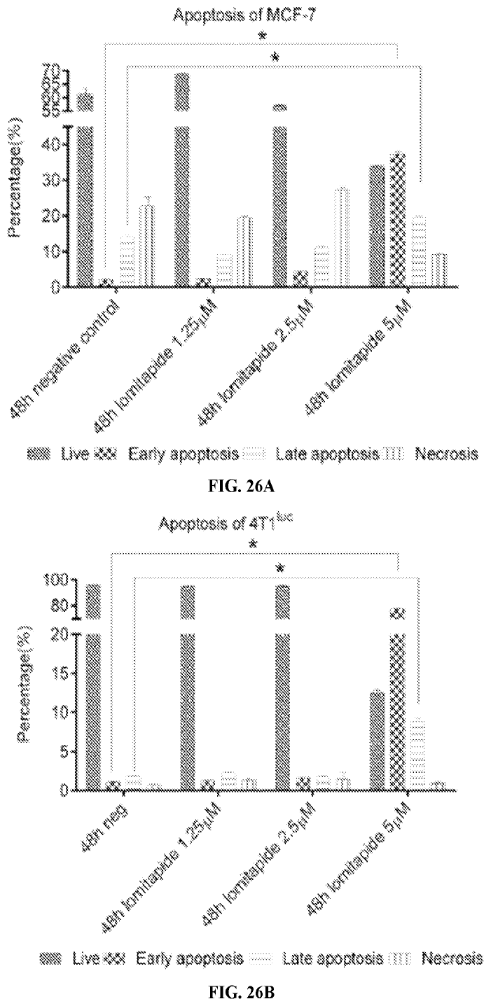

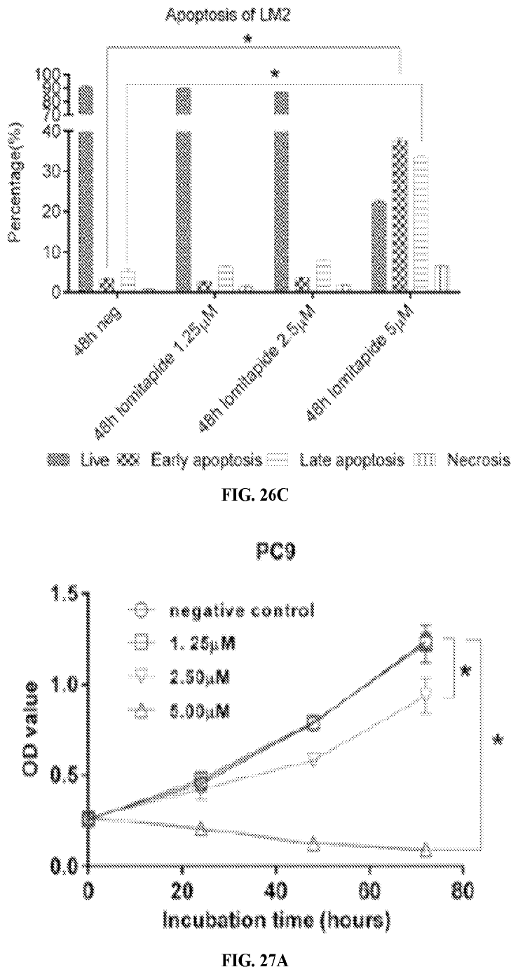

With reference to FIGS. 26A-26C, studies were also conducted to determine the effect of lomitapide treatment on apoptosis of breast cancer cells. Initially, 0.1-0.3 million MCF-7, LM2 or 4T1^{luc} cells were seeded into 6 cm cell culture dishes and cultured overnight. Vehicle control (DMSO) or lomitapide (final concentration: 1.25, 2.5 or 5 μM) was added then into corresponding dishes. After 48 h incubation, the cells were harvested, stained by 7-AAD/APC-annixin V and analyzed by flow cytometry. The results are presented as mean±standard deviation (n=3). * p<0.05, compared to vehicle control (negative control) treatment group. These data show that lomitapide treatment induces apoptosis of breast cancer cells.

### Example 24: Lomitapide Treatment Reduces Lung Cancer Cell Growth

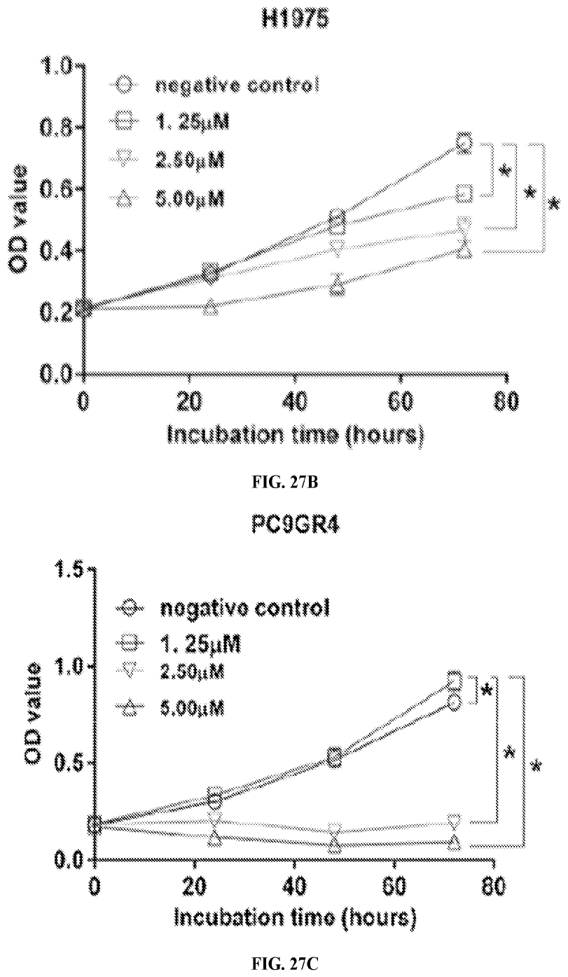

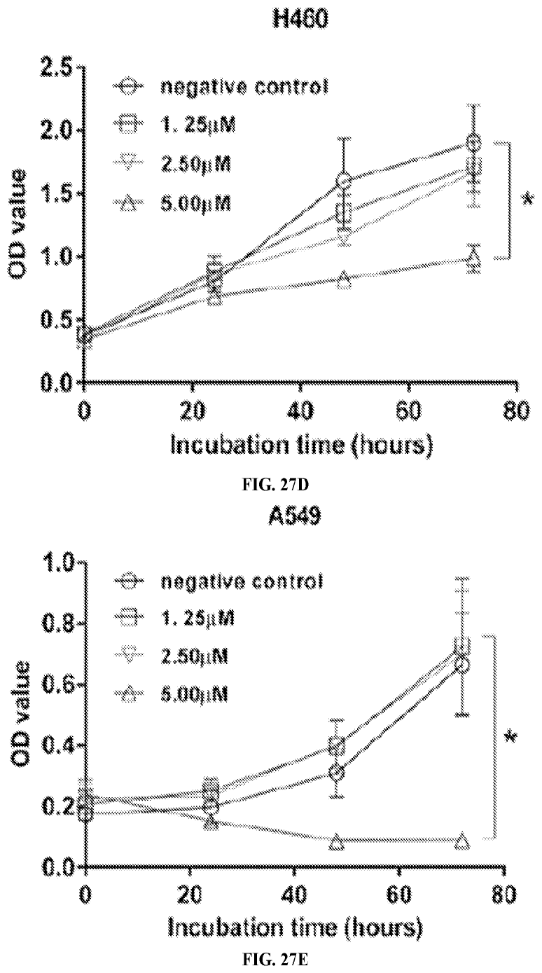

FIGS. 27A-27E include the results of studies to determine the effect of lomitapide treatment on lung cancer cell growth of various cell lines as determined by MTT assay. Initially, 1000 PC9, H1975, PC9GR4, H460, or A549 cells were seeded into 96-well plates. After overnight culture, vehicle (DMSO) or lomitapide (final concentration: 1.25, 2.5 and 5 μM) were added into the corresponding wells. After 24 h, 48 h and 72 h incubation, the culture media were aspirated off and 50 μL of 0.5 mg/mL of MTT containing medium was added into each well. After 3h incubation, 200 μl of DMSO were added into each well. The OD value of each well was read under the wavelength of 570 nm. The results are presented as mean±standard deviation (n=6) * p<0.05, compared to vehicle control (negative control) treatment group. These data show that lomitapide treatment significantly reduces lung cancer cell growth of various cell lines as determined by MTT assay.

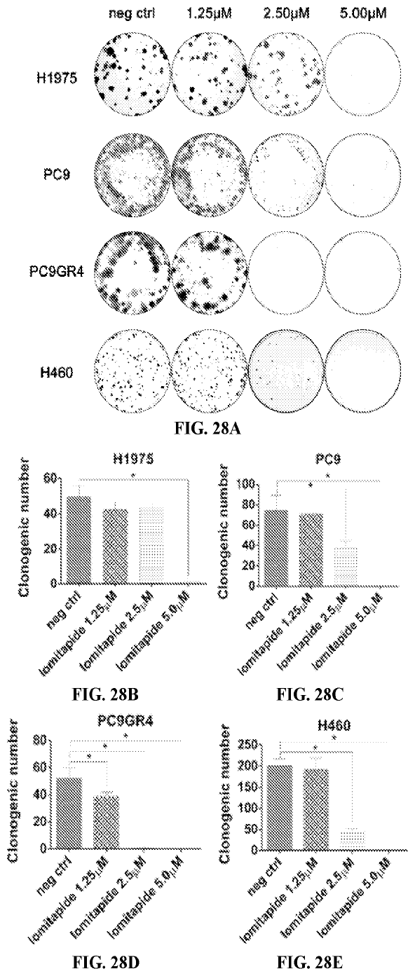

Clonogenic assays were also performed to determine the effect of lomitapide treatment on various lung cancer cell lines, with data reflected in FIGS. 28A-28E. Initially, 100 H1975, PC9, PC9GR4, or H460 cells were seeded into 6-cm cell culture dishes. After overnight culture, vehicle control (DMSO) or lomitapide (final concentration: 1.25, 2.5 or 5 μM) were added into the corresponding dishes. After 72 h culture, the DMSO- or lomitapide-containing media were removed and the fresh culture media were added in followed by additional 10 days culture. The culture media were replaced with fresh media every three days. At the end of cell culture, the final clones in dishes were fixed by 4% paraformaldehyde and stained with crystal violet. FIG. 28A includes representative images from each treatment groups. FIGS. 28B-28E include quantitation of clone numbers (mean±standard deviation, n=3). * p<0.05, compared to vehicle control (negative control) treatment group. These data show that lomitapide treatment significantly reduces lung cancer cell growth as determined by clonogenic assay.

### Example 25: Lomitapide Treatment Induces Apoptosis in Lung Cancer Cells

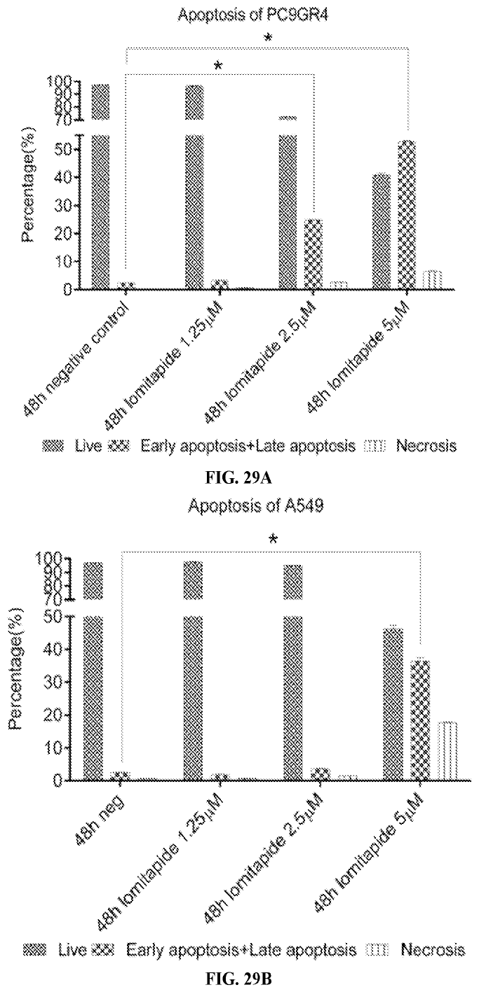

With reference to FIGS. 29A and 29B, studies were also conducted to determine the effect of lomitapide treatment on apoptosis of lung cancer cells. Initially, 0.1-0.3 million PC9GR4 or A549 cells were seeded into 6 cm cell culture dishes and cultured overnight. Vehicle control (DMSO) or lomitapide (final concentration: 1.25, 2.5 or 5 μM) was added then into corresponding dishes. After 48 h incubation, the cells were harvested, stained by 7-AAD/APC-annixin V and analyzed by flow cytometry. The results are presented as mean±standard deviation (n=3). * p<0.05, compared to vehicle control (negative control) treatment group. These data show that lomitapide treatment induces apoptosis of lung cancer cells.

### Example 26: Lomitapide Treatment Reduces Cancer Stem Cell-Like Property of Lung Cancer Cells

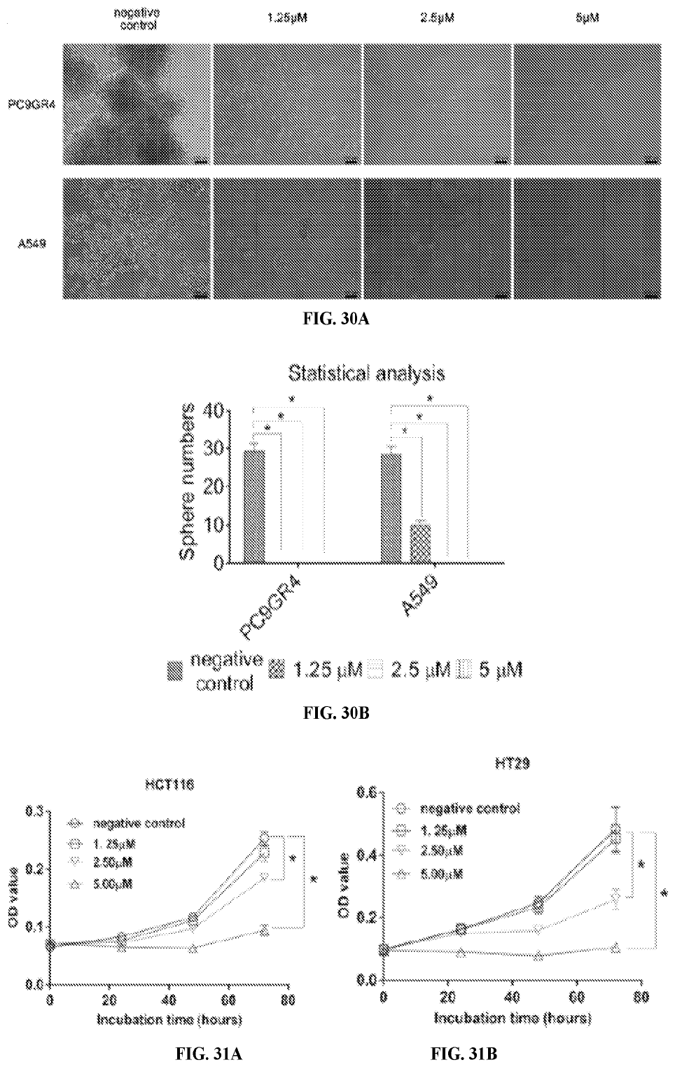

With reference to FIGS. 30A and 30B, studies were also conducted to determine the effect of lomitapide treatment on the cancer stem cell-like property of lung cancer cells. Initially, 2500 lung cancer PC9GR4 or A549 cells were seeded into ultra-adherance 24-well plates; meanwhile, vehicle control (DMSO) or lomitapide (final concentration: 1.25, 2.5 or 5 μM) were added into corresponding wells. After 10-days continuous culture, the final spheres formed were imaged and counted under an inverted phase-contrast microscope. FIG. 30A includes representative sphere images from each treatment groups. FIG. 30B includes the quantitation of sphere numbers (mean±standard deviation, n=3). * p<0.05, compared to vehicle control (negative control) treatment group. These data show that lomitapide treatment significantly reduces cancer stem cell-like property of lung cancer cells.

### Example 27: Lomitapide Treatment Reduces Colon Cancer Cell Growth

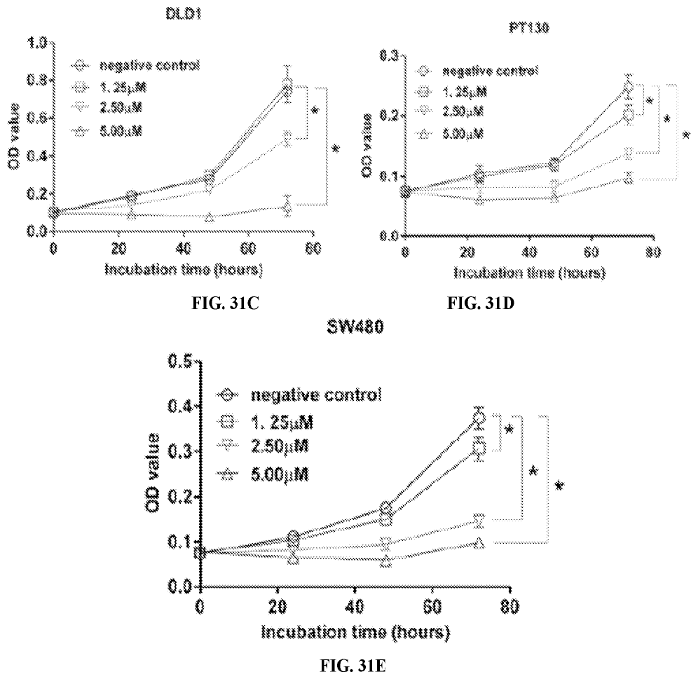

With reference to FIGS. 31A-31F, studies were conducted to determine the effect of lomitapide treatment on colon cancer cell growth as determined by the MTT assay. Initially, 1000 HCT116, HT29, DLD1, PT130, or SW480 cells were seeded into 96-well plates. After overnight culture, vehicle (DMSO) or lomitapide (final concentration: 1.25, 2.5 and 5 μM) were added into the corresponding wells. After 24 h, 48 h and 72 h incubation, the culture media were aspirated off and 50 μL of 0.5 mg/mL of MTT containing medium was added into each well. After 3h incubation, 200 μL of DMSO were added into each well. The OD value of each well was read under the wavelength of 570 nm. The results are presented as mean±standard deviation (n=6) * p<0.05, compared to vehicle control (negative control) treatment group. These data illustrate that lomitapide treatment significantly reduces colon cancer cell growth as determined by MTT assay.

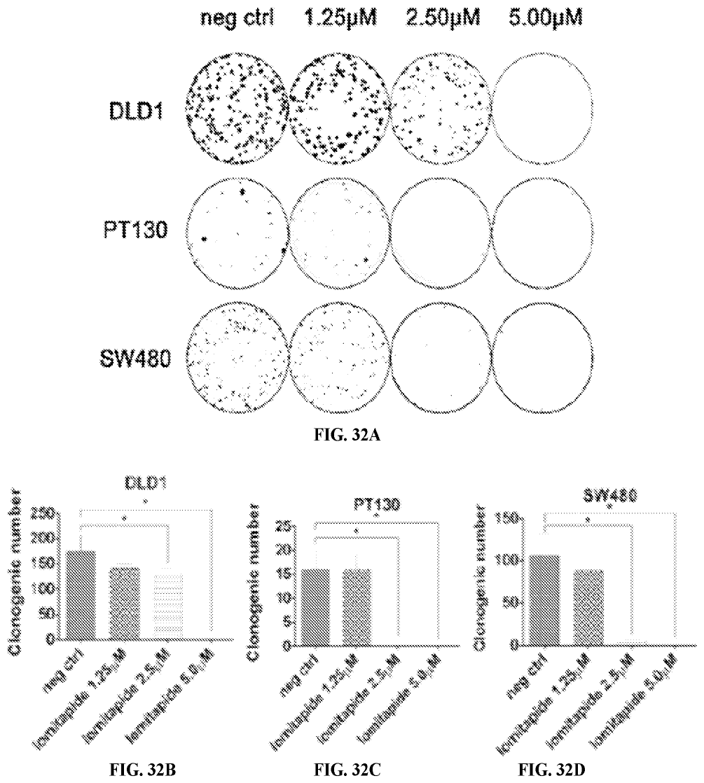

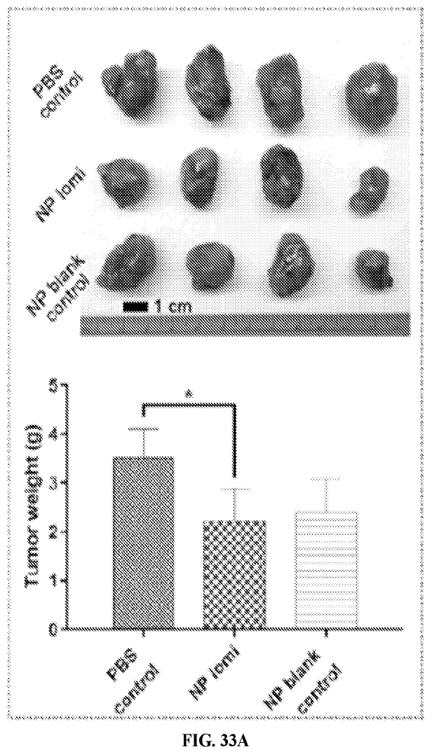

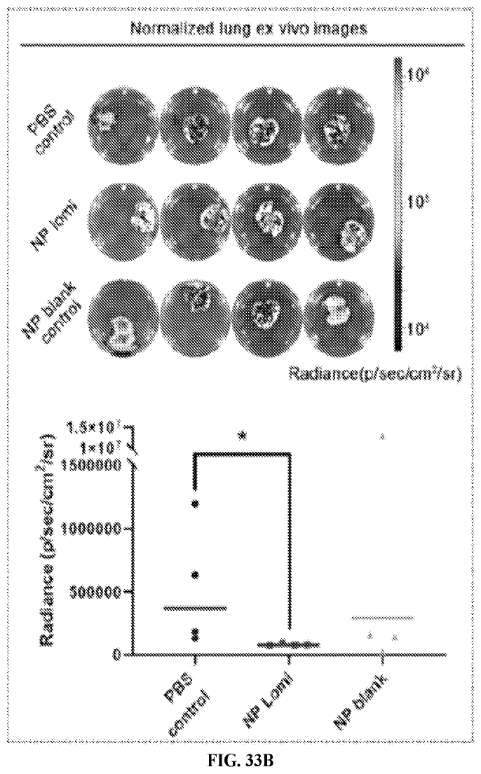

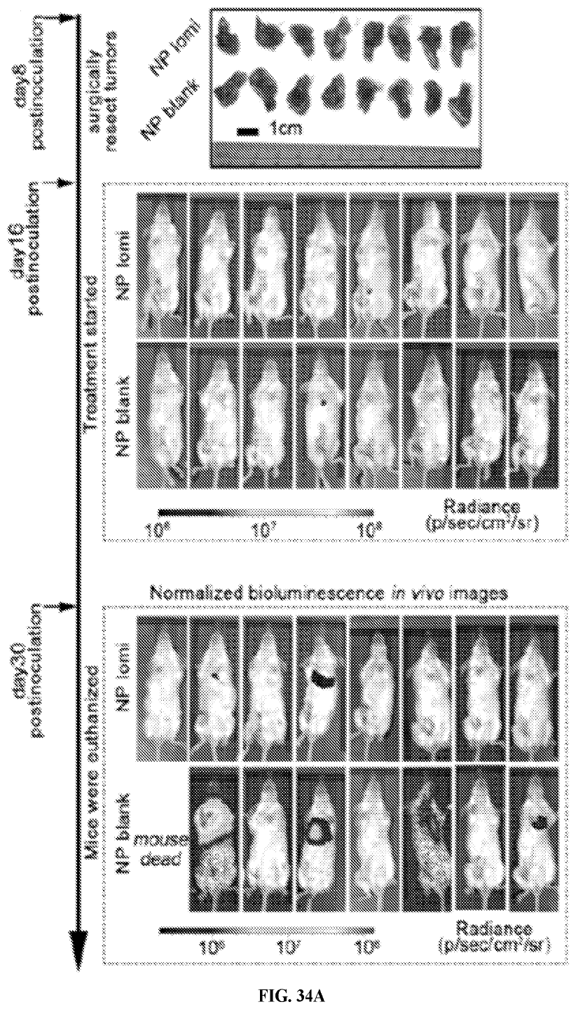

With reference to FIGS. 32A-32D data showing that lomitapide treatment significantly reduces colon cancer cell growth as determined by the clonogenic assay. Initially, 100 DLD1, PT130 or SW480 cells were seeded into 6-cm cell culture dishes. After overnight culture, vehicle control (DMSO) or lomitapide (final concentration: 1.25, 2.5 or 5 μM) were added into the corresponding dishes. After 72 h culture, the DMSO- or lomitapide-containing media were removed and the fresh culture media were added in followed by additional 10 days culture. The culture media were replaced with fresh media every three days. At the end of cell culture, the final clones in dishes were fixed by 4% paraformaldehyde and stained with crystal violet. FIG. 32A includes representative images from each treatment groups. FIGS. 32B-32D include quantitation of clone numbers (mean±standard deviation, n=3). * p<0.05, compared to vehicle control (negative control) treatment group. These data show that lomitapide treatment significantly reduces colon cancer cell growth as determined by clonogenic assay.

### Example 28: Lomitapide Nanoparticle (NP) Treatment Reduces Tumor Growth and Spontaneous Metastasis

In vivo animal studies were conducted and demonstrated that systematic administration of nanoparticle-packaged lomitapide reduces mammary tumor growth and/or spontaneous lung metastasis.

With reference to FIGS. 33A and 33B, one million human TNBC LM2 cells were inoculated into nude mouse 4^{th} mammary fat pad to produce orthotopic mammary tumors. When mammary tumor size reached approximately 300 mm³, tumor bearing mice were divided into three groups and each group of mice had similar tumor sizes. Mice were treated thrice per week for 5 weeks with PBS (control), nanoparticle-packaged lomitapide (NP lomi, 10 mg/kg body weight) or nanoparticle blank (NP blank control) via tail vein injection. At the end of treatment, mice were euthanized, and lungs were imaged using the IVIS imaging system to examine lung metastasis. FIG. 33A includes images of mouse mammary xenograft tumors and their weight extracted from mice treated with PBS, nanoparticle-packaged lomitapide or NP blank, respectively (mean±SD, n=4). * p<0.05. FIG. 33 B includes normalized ex vivo bioluminescence images of lungs harvested from the mice of each group and the bioluminescence signal intensities from the ex vivo lung images (mean±SD, n=4). * p<0.05. These data show that systematic administration of nanoparticle-packaged lomitapide significantly reduces mouse orthotopic mammary tumor growth and spontaneous lung metastasis.

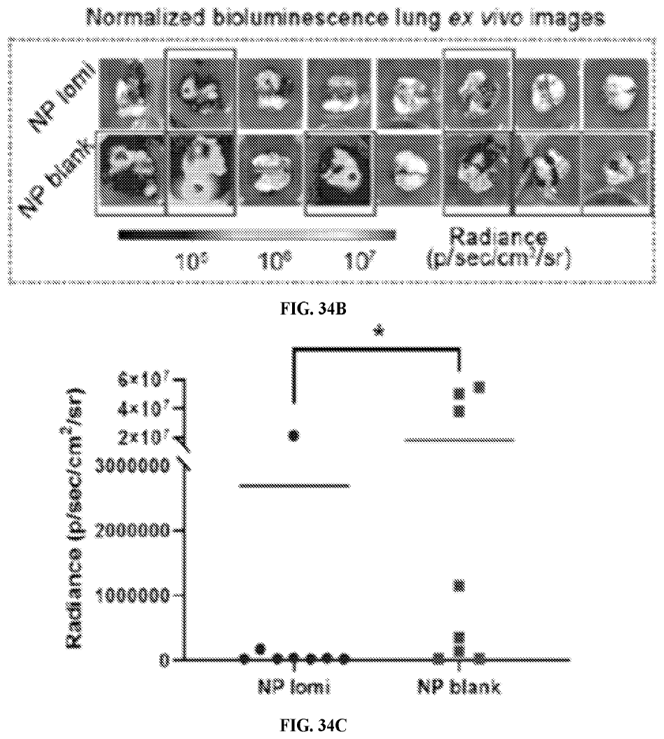

FIGS. 34A-34C include data from additional animal studies. With reference to FIG. 34A, zero point three million of mouse breast cancer 4T1^{luc} cells were inoculated into the 4^{th} mammary fat pad of Balb/c mouse to produce mammary tumors. When tumor sizes reached about 300 mm³, mammary tumors were surgically removed at the 8th day post 4T1 cell inoculation. At the 16th day post 4T1^{luc} cell inoculation, all mice were imaged by the IVIS imaging instrument to confirm the complete eradication of primary mammary tumor. Primary mammary tumor-free mice were randomly divided into two groups: one group of mice were then treated with nanoparticle-packaged lomitapide (NP lomi, 10 mg/kg body weight) and the other group were treated with blank nanoparticle (NP blank) two time per week for two weeks via tail vein injection. At the end of treatment (the 30th day post 4T1^{luc} cell inoculation), all mice were first imaged by the IVIS imaging instrument to detect spontaneous lung metastasis. With reference to FIG. 34B, after imaging, all mice were euthanized and lungs from both groups of mice were collected for further ex vivo IVIS imaging analysis. Six out of eight mice in NP blank-treated group developed lung metastasis; however, only two of eight mice in nanoparticle-packaged lomitapide treatment group developed lung metastasis. With reference to FIG. 34C, the lung bioluminescent intensities are significantly stronger in NP blank treatment group than that in nanoparticle-packaged lomitapide (NP lomi) treatment group (mean±SD, n=8). * p<0.05. These data show that systematic administration of nanoparticle-packaged lomitapide significantly reduces mammary tumor growth and spontaneous lung metastasis in a syngeneic mouse model.

### Example 29: Synergistic Results with Combined Lomitapide (Lomi) and Paclitaxel (PTX) Treatment on Growth of Cancer Cells

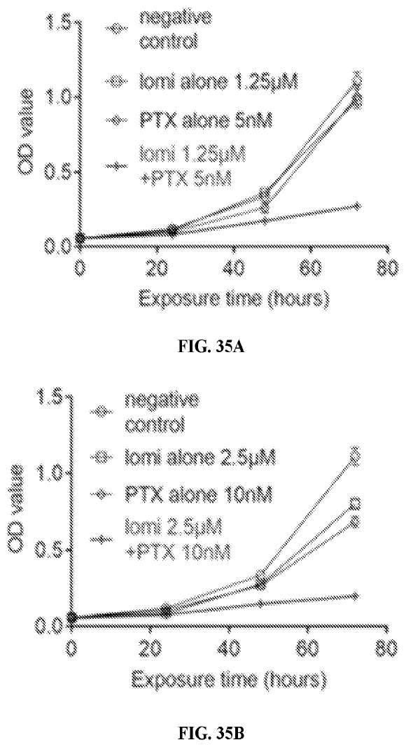

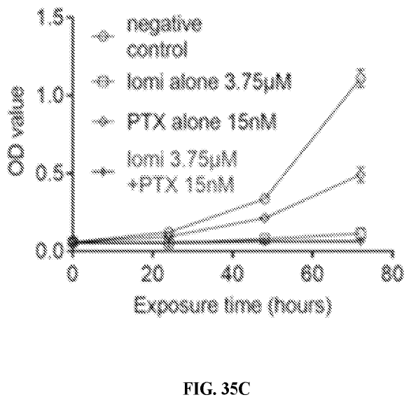

FIGS. 35A-35C include results showing the synergistic inhibitory effect of combined lomitapide (lomi) and paclitaxel (PTX) treatment on breast cancer cell growth at various doses.

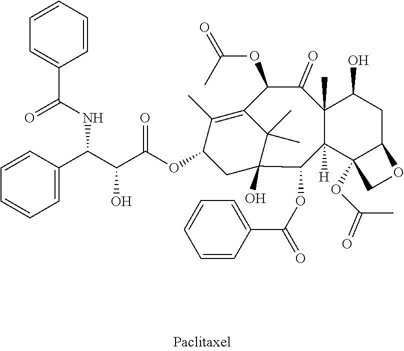 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

One thousand 4T1 cells were seeded into 96-well plates; at the 2nd day, different concentrations of vehicle control, lomi, PTX or the combination of lomi and PTX were added into the culture media and incubated for 24 hours. The cell growth was assessed by the MTT assay.

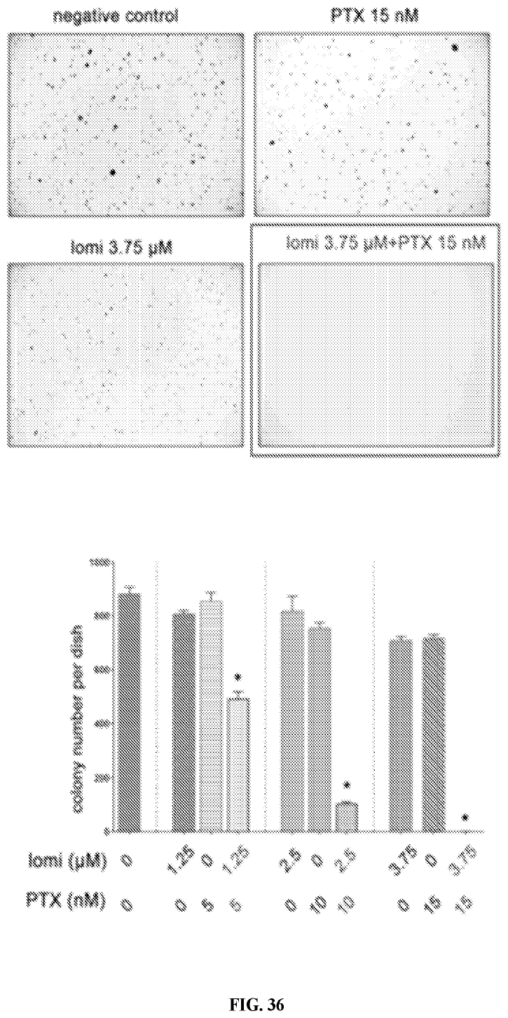

FIG. 36 also includes data showing the synergistic inhibitory effect of combined lomitapide (lomi) and paclitaxel (PTX) treatment on breast cancer cell growth in soft agar. After the 2^{nd} day of 1000 TNBC 4T1 cells being seeded into the upper layer agar, PTX and lomi were added into the surface culture media, and 4T1 cells were continuously cultured for 30 more days. These data show that, as compared to individual administration, administration of a combination of lomatapide and paclitaxel, at various doses, synergistically effect growth of cancer cells.

### Example 30: Synergistic Results with Combined Lomitapide (NP-Lomi) and Paclitaxel (PTX) Treatment on Breast Tumor Growth and Metastasis

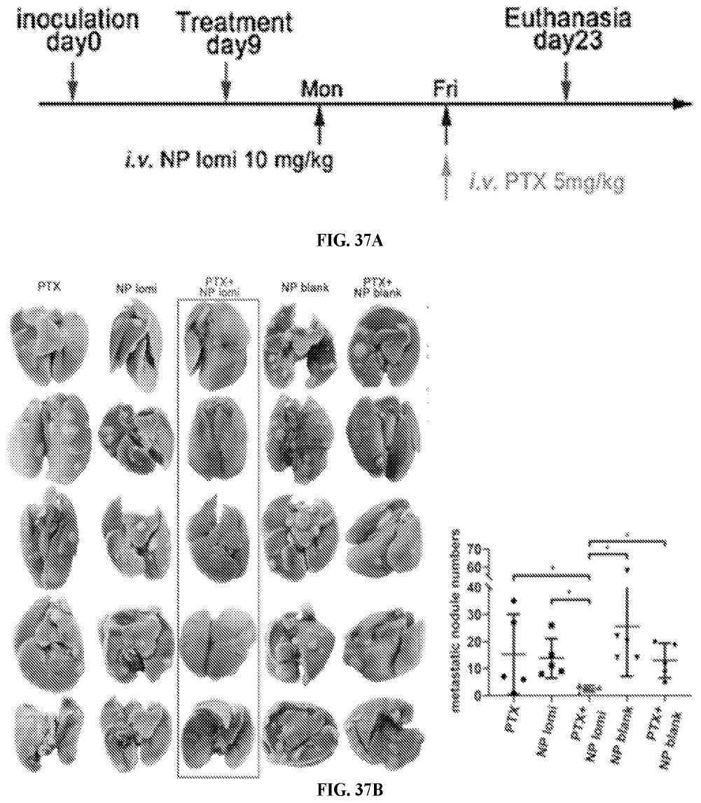

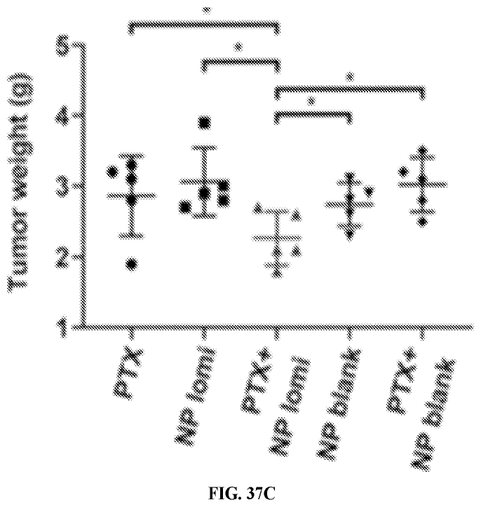

FIGS. 37A-37C include data showing a synergistic inhibitory effect of combined nanoparticle-packaged lomitapide (NP lomi) and paclitaxel (PTX) treatment on breast tumor growth and metastasis. FIG. 37A illustrates the treatment scheme on syngeneic 4T1 tumor model. FIG. 37B includes ex vivo images of lungs with metastatic nodules as well as their statistics. (n=5, * p<0.05, unpaired nonparametric one-way ANOVA analysis of Kruskal-Wallis test). FIG. 37C includes a weight comparison of the collected 4T1 primary tumors among groups. (n=5, * p<0.05, one-tailed unpaired nonparametric t test of Mann-Whitney test). These data show that, as compared to individual administration, administration of a combination of NP-lomatapide and paclitaxel, at various doses, synergistically effect tumor growth and metastasis.

It will be understood that various details of the presently disclosed subject matter can be changed without departing from the scope of the subject matter disclosed herein. Furthermore, the foregoing description is for the purpose of illustration only, and not for the purpose of limitation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. G. Bianchini, J. M. Balko, I. A. Mayer, M. E. Sanders, L. Gianni, Triple-negative breast cancer: challenges and opportunities of a heterogeneous disease, Nat. Rev. Clin. Oncol. 13 (2016) 674-690.
2. M. Balic, H. Lin, L. Young, D. Hawes, A. Giuliano, G. McNamara, R. H. Datar, R. J. Cote, Most early disseminated cancer cells detected in bone marrow of breast cancer patients have a putative breast cancer stem cell phenotype, Clin. Cancer Res. 12 (2006) 5615-5621.
3. M. Ksigzkiewicz, A. Markiewicz, A. J. Zaczek, Epithelial-mesenchymal transition: a hallmark in metastasis formation linking circulating tumor cells and cancer stem cells, Pathobiology 79 (2012) 195-208.
4. U. De Giorgi, V. Valero, E. Rohren, S. Dawood, N. T. Ueno, M. C. Miller, G. V. Doyle, S. Jackson, E. Andreopoulou, B. C. Handy, Circulating tumor cells and [18F] fluorodeoxyglucose positron emission tomography/computed tomography for outcome prediction in metastatic breast cancer, J. Clin. Oncol. 27 (2009) 3303-3311.
5. B. Rack, C. Schindlbeck, J. Juckstock, U. Andergassen, P. Hepp, T. Zwingers, T. W. P. Friedl, R. Lorenz, H. Tesch, P. A. Fasching, T. Fehm, A. Schneeweiss, W. Lichtenegger, M. W. Beckmann, K. Friese, K. Pantel, W. Janni, T. S. S. Group, Circulating tumor cells predict survival in early average-to-high risk breast cancer patients, JNCI: J. Nat. Cancer Inst. 106 (2014).
6. J. P. Hong, S. H. Sun, M. Ben-Nakhi, Modified superficial circumflex iliac artery perforator flap and supermicrosurgery technique for lower extremity reconstruction: a new approach for moderate-sized defects, Ann. Plast. Surg. 71 (2013) 380-383.
7. S. J. Isakoff, Triple negative breast cancer: role of specific chemotherapy agents, Cancer J. (Sudbury, Mass.) 16 (2010) 53.
8. C. Liedtke, C. Mazouni, K. R. Hess, F. Andre, A. Tordai, J. A. Mejia, W. F. Symmans, A. M. Gonzalez-Angulo, B. Hennessy, M. Green, Response to neoadjuvant therapy and long-term survival in patients with triple-negative breast cancer, J. Clin. Oncol. 26 (2008) 1275-1281.
9. Y.-P. Liu, C.-J. Yang, M.-S. Huang, C.-T. Yeh, A. T. Wu, Y.-C. Lee, T.-C. Lai, C.-H. Lee, Y.-W. Hsiao, J. Lu, Cisplatin selects for multidrug-resistant CD133+ cells in lung adenocarcinoma by activating notch signaling, Cancer Res. 73 (2013) 406-416.
10. B. A. Hilton, Z. Li, P. R. Musich, H. Wang, B. M. Cartwright, M. Serrano, X. Z. Zhou, K. P. Lu, Y. Zou, ATR plays a direct antiapoptotic role at mitochondria, which is regulated by prolyl isomerase Pin1, Mol. Cell 60 (2015) 35-46.
11. V. Ilio, M. Gwenola, M. Ruggero De, K. Guido, G. Lorenzo, DNA damage in stem cells, Mol. Cell 66 (2017) 306-319.
12. T. M. Phillips, W. H. McBride, F. Pajonk, The response of CD24-/low/CD44+ breast cancer-initiating cells to radiation, J. Natl. Cancer Inst. 98 (2006) 1777-1785.
13. C. Gong, B. Liu, Y. Yao, S. Qu, W. Luo, W. Tan, Q. Liu, H. Yao, L. Zou, F. Su, Potentiated DNA damage response in circulating breast tumor cells confers resistance to chemotherapy, J. Biol. Chem. 290 (2015) 14811-14825.
14. S. Bao, Q. Wu, R. E. McLendon, Y. Hao, Q. Shi, A. B. Hjelmeland, M. W. Dewhirst, D. D. Bigner, J. N. Rich, Glioma stem cells promote radioresistance by preferential activation of the DNA damage response, Nature 444 (2006) 756.
15. L. Kelland, The resurgence of platinum-based cancer chemotherapy, Nat. Rev. Cancer 7 (2007) 573.
16. J. M. Reichert, A guide to drug discovery: trends in development and approval times for new therapeutics in the United States, Nat. Rev. Drug Discov. 2 (2003) 695.
17. J. Gilbert, P. Henske, A. Singh, Rebuilding big pharma's business model, Vivo-New York then Norwalk, 21 2003, pp. 73-80.
18. Y. Li, Y. Xiao, H.-P. Lin, D. Reichel, Y. Bae, E. Y. Lee, Y. Jiang, X. Huang, C. Yang, Z. Wang, In vivo β-catenin attenuation by the integrin α5-targeting nano-delivery strategy suppresses triple negative breast cancer stemness and metastasis, Biomaterials 188 (2019) 160-172.
19. B. Humphries, Z. Wang, Y. Li, J.-R. Jhan, Y. Jiang, C. Yang, ARHGAP18 down-regulation by miR-200b suppresses metastasis of triple-negative breast cancer by enhancing activation of RhoA, Cancer Res. 77 (15) (2017) 4051-4064.
20. A. J. Minn, G. P. Gupta, P. M. Siegel, P. D. Bos, W. Shu, D. D. Giri, A. Viale, A. B. Olshen, W. L. Gerald, J. Massague, Genes that mediate breast cancer metastasis to lung, Nature 436 (2005) 518.
21. B. Humphries, Z. Wang, A. L. Oom, T. Fisher, D. Tan, Y. Cui, Y. Jiang, C. Yang, MicroRNA-200b targets protein kinase Ca and suppresses triple-negative breast cancer metastasis, Carcinogenesis 35 (2014) 2254-2263.
22. G. Ruan, A. Agrawal, A. I. Marcus, S. Nie, Imaging and tracking of tat peptide-conjugated quantum dots in living cells: new insights into nanoparticle uptake, intracellular transport, and vesicle shedding, J. Am. Chem. Soc. 129 (2007) 14759-14766.
23. S. Barua, S. Mitragotri, Synergistic targeting of cell membrane, cytoplasm, and nucleus of cancer cells using rod-shaped nanoparticles, ACS Nano 7 (2013) 9558-9570.
24. W. E. Ford, O. Harnack, A. Yasuda, J. M. Wessels, Platinated DNA as precursors to templated chains of metal nanoparticles, Adv. Mater. 13 (2001) 1793-1797.
25. A. Kumar, M. Pattarkine, M. Bhadbhade, A. B. Mandale, K. N. Ganesh, S. S. Datar, C. V. Dharmadhikari, M. Sastry, Linear superclusters of colloidal gold particles by electrostatic assembly on DNA templates, Adv. Mater. 13 (2001) 341-344.
26. C. M. Goodman, N. S. Chari, G. Han, R. Hong, P. Ghosh, V. M. Rotello, DNA-binding by functionalized gold nanoparticles: mechanism and structural requirements, Chem. Biol. Drug Des. 67 (2006) 297-304.
27. K. K. Sandhu, C. M. McIntosh, J. M. Simard, S. W. Smith, V. M. Rotello, Gold nano-particle-mediated transfection of mammalian cells, Bioconjug. Chem. 13 (2002) 3-6.
28. D. Zanchet, C. M. Micheel, W. J. Parak, D. Gerion, A. P. Alivisatos, Electrophoretic isolation of discrete Au nanocrystal/DNA conjugates, Nano Lett. 1 (2001) 32-35.
29. Y. Liu, W. Meyer-Zaika, S. Franzka, G. Schmid, M. Tsoli, H. Kuhn, Gold-cluster degradation by the transition of B-DNA into A-DNA and the formation of nanowires, Angew. Chem. Int. Ed. 42 (2003) 2853-2857.
30. S. M. Aris, K. M. Knott, X. Yang, D. A. Gewirtz, N. P. Farrell, Modulation of trans-planaramine platinum complex reactivity by systematic modification of carrier and leaving groups, Inorg. Chim. Acta 362 (2009) 929-934.
31. C. Redon, D. Pilch, E. Rogakou, O. Sedelnikova, K. Newrock, W. Bonner, Histone H2a variants H2AX and H2AZ, Curr. Opin. Genet. Dev. 12 (2002) 162-169.
32. S. M. Nolte, C. Venugopal, N. McFarlane, O. Morozova, R. M. Hallett, E. O'farrell, B. Manoranjan, N. K. Murty, P. Klurfan, E. Kachur, A cancer stem cell model for studying brain metastases from primary lung cancer, J. Natl. Cancer Inst. 105 (2013) 551-562.
33. Y. Li, M. Xian, B. Yang, M. Ying, Q. He, Inhibition of KLF4 by statins reverses Adriamycin-induced metastasis and cancer stemness in osteosarcoma cells, Stem Cell Rep. 8 (2017) 1617-1629.
34. M. G. Krebs, R. Sloane, L. Priest, L. Lancashire, J.-M. Hou, A. Greystoke, T. H. Ward, R. Ferraldeschi, A. Hughes, G. Clack, Evaluation and prognostic significance of circulating tumor cells in patients with non-small-cell lung cancer, J. Clin. Oncol. 29 (2011) 1556-1563.
35. J. S. De Bono, H. I. Scher, R. B. Montgomery, C. Parker, M. C. Miller, H. Tissing, G. V. Doyle, L. W. Terstappen, K. J. Pienta, D. Raghavan, Circulating tumor cells predict survival benefit from treatment in metastatic castration-resistant prostate cancer, Clin. Cancer Res. 14 (2008) 6302-6309.
36. B. Rack, C. Schindlbeck, J. Juckstock, U. Andergassen, P. Hepp, T. Zwingers, T. W. Friedl, R. Lorenz, H. Tesch, P. A. Fasching, Circulating tumor cells predict survival in early average-to-high risk breast cancer patients, J. Natl. Cancer Inst. 106 (2014) (dju066).
37. M. Cristofanilli, G. T. Budd, M. J. Ellis, A. Stopeck, J. Matera, M. C. Miller, J. M. Reuben, G. V. Doyle, W. J. Allard, L. W. Terstappen, Circulating tumor cells, disease progression, and survival in metastatic breast cancer, N. Engl. J. Med. 351 (2004) 781-791.
38. M. Becker, A. Nitsche, C. Neumann, J. Aumann, I. Junghahn, I. Fichtner, Sensitive PCR method for the detection and real-time quantification of human cells in xe-notransplantation systems, Br. J. Cancer 87 (2002) 1328.
39. W. P. Roos, A. D. Thomas, B. Kaina, DNA damage and the balance between survival and death in cancer biology, Nat. Rev. Cancer 16 (2016) 20.
40. E. Porcel, S. Liehn, H. Remita, N. Usami, K. Kobayashi, Y. Furusawa, C. Le Sech, S. Lacombe, Platinum nanoparticles: a promising material for future cancer therapy?Nanotechnology 21 (2010) 085103.
41. J. Gao, G. Liang, B. Zhang, Y. Kuang, X. Zhang, B. Xu, FePt@ CoS2 yolk-shell nanocrystals as a potent agent to kill HeLa cells, J. Am. Chem. Soc. 129 (2007) 1428-1433.
42. P. Asharani, N. Xinyi, M. P. Hande, S. Valiyaveettil, DNA damage and p53-mediated growth arrest in human cells treated with platinum nanoparticles, Nanomedicine 5 (2010) 51-64.
43. H. S. Choi, W. Liu, P. Misra, E. Tanaka, J. P. Zimmer, B. I. Ipe, M. G. Bawendi, J. V. Frangioni, Renal clearance of quantum dots, Nat. Biotechnol. 25 (2007) 1165.
44. Y. Ming, Y. Li, H. Xing, M. Luo, Z. Li, J. Chen, J. Mo, S. Shi, Circulating tumor cells: from theory to nanotechnology-based detection, Front. Pharmacol. 8 (2017) 35.
45. P. U. Atukorale, S. P. Raghunathan, V. Raguveer, T. J. Moon, C. Zheng, P. A. Bielecki, M. L. Wiese, A. L. Goldberg, G. Covarrubias, C. J. Hoimes, Nanoparticle encapsulation of synergistic immune agonists enables systemic codelivery to tumor sites and IFNβ-driven antitumor immunity, Cancer Res. 79 (2019) 5394-5406.
46. H. Du, S. Zhao, Y. Wang, Z. Wang, B. Chen, Y. Yan, Q. Yin, D. Liu, F. Wan, Q. Zhang, Y. Wang, pH/Cathepsin B hierarchical-responsive nanoconjugates for enhanced tumor penetration and chemo-immunotherapy, Adv. Funct. Mater. 30 (2020) 2003757.
47. J. Shi, P. W. Kantoff, R. Wooster, O. C. Farokhzad, Cancer nanomedicine: progress, challenges and opportunities, Nat. Rev. Cancer 17 (2017) 20.
48. R. Tong, L. Yala, T. M. Fan, J. Cheng, The formulation of aptamer-coated paclitax-el-polylactide nanoconjugates and their targeting to cancer cells, Biomaterials 31 (2010) 3043-3053.
49. B. Felding-Habermann, T. E. O'Toole, J. W. Smith, E. Fransvea, Z. M. Ruggeri, M. H. Ginsberg, P. E. Hughes, N. Pampori, S. J. Shattil, A. Saven, Integrin activation controls metastasis in human breast cancer, Proc. Natl. Acad. Sci. 98 (2001) 1853-1858.

It will be understood that various details of the presently disclosed subject matter can be changed without departing from the scope of the subject matter disclosed herein. Furthermore, the foregoing description is for the purpose of illustration only, and not for the purpose of limitation.

1. A composition for treating cancer in a subject, comprising: miriplatin assembled into an ultrasmall dot (uPtD).

2. The composition of claim 1, wherein the uPtD is encapsulated in a nanoparticle.

3. The composition of claim 2, wherein the nanoparticle is a lipid-polymer hybrid (LPH) nanoparticle.

4. The composition of claim 2, wherein an exterior layer of the nanoparticle is anchored with a polar species.

5. The composition of claim 4, wherein the polar species is PEG₂₀₀₀.

6. The composition of claim 2, wherein the nanoparticle is decorated with a targeting agent.

7. The composition of claim 6, wherein the targeting agent is RGD for targeting triple-negative breast cancer (TNBC) cells.

8. A composition for treating cancer in a subject, comprising: lomitapide or a pharmaceutically-acceptable salt thereof in an effective amount for inhibiting the growth of and/or killing a cancer cell.

9. The composition of claim 8, and further comprising paclitaxel or a pharmaceutically-acceptable salt thereof.

10. The composition of claim 8, and further comprising a nanoparticle, wherein the lomitapide or a pharmaceutically-acceptable salt thereof is packaged within the nanoparticle.

11. The composition of claim 10, wherein the nanoparticle is a lipid-polymer hybrid (LPH) nanoparticle.

12. The composition of claim 11, wherein an exterior layer of the nanoparticle is anchored with a polar species.

13. The composition of claim 11, wherein the nanoparticle is decorated with a targeting agent.

14. (canceled)

15. A method of inhibiting the growth of and/or killing a cancer cell, comprising: administering an effective amount of the composition of claim 1, thereby inhibiting the growth of and/or killing the cancer cell.

16. The method of claim 15, wherein the cancer cell is a breast cancer cell, a lung cancer cell, or a colon cancer cell.

17. The method of claim 15, wherein the breast cancer cell is a triple-negative breast cancer (TNBC) cell.

18-19. (canceled)

20. The method of claim 15, wherein the cancer cell is in a subject.

21-27. (canceled)

28. The method of claim 15, comprising administering the composition by injection.

29. The method of claim 28, wherein the composition comprises a platinum (II) genotoxic agents (Pt(II)) packaged within an ultra-small nanoparticle.

30. The method of claim 15, comprising orally-administering the composition.

31-36. (canceled)
